(19) United States
(12) Patent Application Publication
(10) Pub. No.: US 2011/0097713 A1
(43) Pub. Date: Apr. 28, 2011

(54) Compositions and methods for pharmacogenomic screening of CYP2C9 and VKORC1

(75) Inventors: Thomas Briggs, Nazareth, PA (US); James R. Johnson, Gainesville, GA (US); Jeremy C. Pridgen, Cary, NC (US); Michael P. Murphy, Raleigh, NC (US); Suzanne L. Phillips, Ganer, NC (US); Pamela J. Nakhle, Raleigh, NC (US)

(73) Assignee: Catalyst Assets LLC

(21) Appl. No.: 12/583,539

(22) Filed: Aug. 21, 2009

Related U.S. Application Data
(63) Continuation of application No. PCT/US2008/054994, filed on Feb. 26, 2008.
(60) Provisional application No. 60/903,778, filed on Feb. 27, 2007.

(51) Int. Cl. C12Q 1/68; C07H 21/00

(52) U.S. Cl. 435/6; 536/23.1

(57) ABSTRACT

Compositions and methods for determining an optimal dose of a medication for a subject are described that include determining the subject's genotype for the CYP2C9 and VKORC1 genes and determining the dose of the medication based on the genotype. Articles of manufacture also are provided that include polynucleotides for genotyping.

### CROSS REFERENCE TO RELATED APPLICATIONS

This present invention is a continuation patent application that claims priority to PCT patent application number PCT/US2008/054994, filed on Feb. 26, 2008, which claims the benefit of U.S. Application 60/903,778, filed on Feb. 27, 2007, the entirety of which are herein incorporated by reference.

### FIELD OF THE INVENTION

This invention relates to compositions and methods for selecting an optimal dose for a medication (e.g., warfarin) for a subject, and more particularly for selecting the subject's optimal dose based on the genotype of genes including genes including cytochrome P450 2C9 (CYP2C9) and vitamin K epoxide reductase complex subunit 1 (VKORC1).

### BACKGROUND OF INVENTION

Pharmacogenomics is the study of inheritable traits affecting subject response to drug treatment. Differential responses to drug treatment may be due to underlying genetic polymorphisms (genetic variations sometimes called mutations) that affect drug metabolism. Testing subjects for these genetic polymorphisms may help to prevent adverse drug reactions and facilitate appropriate drug dosing regimens.

In the clinical setting, pharmacogenomics may enable physicians to select the individual subject. That is, pharmacogenomics can identify those subjects with the right genetic makeup to respond to a given therapy, and also can identify those subjects with genetic variations in the genes that control the metabolism of pharmaceutical compounds, so that the proper dosage can be administered.

CYP2C9 is a drug-metabolizing enzyme that catalyzes the biotransformation of many other clinically useful drugs, such as angiotensin II blockers, nonsteroidal anti-inflammatory drugs, the alkylating anticancer prodrugs, sulfonylureas, some antidepressants, tamoxifen, and many others. Of special interest are those drugs with narrow therapeutic indices, such as S-warfarin, tolbutamide, and phenytoin, where impairment in CYP2C9 metabolic activity might cause difficulties in dose adjustment as well as toxicity. Individuals identified using the screening methods as having CYP2C9 poor metabolizer variants tend to exhibit different pharmacokinetics (drug levels) than normal individuals.

Coumarin derivatives such as warfarin represent the therapy of choice for the long-term treatment and prevention of thromboembolic events. These agents target blood coagulation by inhibiting VKORC1. Certain mutations in VKORC1 are associated with resistance to coumarin-type anticoagulant drugs (a.k.a. warfarin resistance).

Genomic testing of genes encoding CYP2C9 and VKORC1 not only provides rational drug selection and drug dosing but it also provides a safe method by which potentially dangerous side effects can be avoided in a subject in need of a particular medication. There is a need for compositions and methods for genotyping CYP2C9 and VKORC1.

It would be advantageous to have compositions and methods for genotyping CYP2C9 and VKORC1. The present invention provides such compositions and methods.

### SUMMARY OF THE INVENTION

There is now provided an isolated polynucleotide comprising a nucleotide sequence or complement thereof, wherein the nucleotide sequence is selected from the group consisting of:

(SEQ ID NO: 1)
5′-CCCCTGAATTGCTACAACAAATGTG-3′;
(SEQ ID NO: 2)
5′-GACTTCGAAAACATGGAGTGCA-3′;
(SEQ ID NO: 3)
5′-CAGAGATACCTTGACCTTC-3′;
(SEQ ID NO: 4)
5′-CCAGAGATACATTGACCTTC-3′;
(SEQ ID NO: 5)
5′-CTCATGACGCTGCGGAATTT-3′;
(SEQ ID NO: 6)
5′-GAAGATAGTAGTCCAGTAAGGTCAGTGATATG-3′;
(SEQ ID NO: 7)
5′-CATTGAGGACCGTGTTCA-3′;
(SEQ ID NO: 8)
5′-CATTGAGGACTGTGTTCAA-3′;
(SEQ ID NO: 9)
5′-ATCCTGACGTGGCCAAAGG-3′;
(SEQ ID NO: 10)
5′-CCACCTGGGCTATCCTCTGTT-3′;
(SEQ ID NO: 11)
5′-CCAGGAGATCATCGACCCTTGGAC-3′;
(SEQ ID NO: 12)
5′-CCAGGAGATCATCGACTCTTGGACTAGG-3′;
(SEQ ID NO: 13)
5′-CCCTAGATGTGGGGCTTCTAGATTA-3′;
(SEQ ID NO: 14)
5′-AGCGTGTGGCACATTTGGT-3′;
(SEQ ID NO: 15)
5′-CTCCTGCCATACCCACACATGACAAT-3′;
(SEQ ID NO: 16)
5′-CTCCTGCCATACCCGCACATGA-3′;
(SEQ ID NO: 17)
5′-GACTTCGAAAACATGGAGTTGCA-3′,
(SEQ ID NO: 18)
5′-TGCAAGACAGGAGCCACATG-3′,
(SEQ ID NO: 19)
5′-TTACCTTGGGAATGAGATAGTTTCTG-3′,
(SEQ ID NO: 20)
5′-TCCAGAGATACCTTGACCTTCTC-3′,
(SEQ ID NO: 21)
5′-TCCAGAGATACATTGACCTTCTC-3′,

wherein the isolated polynucleotide has a total nucleotide length of about 18 to about 50 nucleotides.

The isolated polynucleotides are useful as primers and/or probes for detecting single nucleotide polymorphisms (SNPs) in subjects, particularly SNPs in the CYP2C9 and VKORC1 genes.

In another aspect, the present invention provides an isolated polynucleotide comprising a sequence consisting of at least 10, at least 18, at least 20, or at least 50 contiguous nucleotides or a complement thereof, wherein the contiguous nucleotides are contained in a nucleotide sequence selected from the group consisting of (SEQ ID NO: 1), (SEQ ID NO: 2), (SEQ ID NO: 3), (SEQ ID NO: 4), (SEQ ID NO: 5), (SEQ ID NO: 6), (SEQ ID NO: 7), (SEQ ID NO: 8), (SEQ ID NO: 9), (SEQ ID NO: 10), (SEQ ID NO: 11), (SEQ ID NO: 12), (SEQ ID NO: 13), (SEQ ID NO: 14), (SEQ ID NO: 15), (SEQ ID NO: 16), (SEQ ID NO: 17), (SEQ ID NO: 18), (SEQ ID NO: 19), (SEQ ID NO: 20), and (SEQ ID NO: 21).

In other aspects, the present invention provides a set of primers comprising at least one primer pair. The at least one primer pair comprises a first primer and a second primer each having a sequence consisting of at least 10, at least 18, at least 20, or at least 50 contiguous nucleotides or a complement thereof, wherein the contiguous nucleotides are, respectively, contained in a nucleotide sequence selected from the group consisting of

(SEQ ID NO: 1) and (SEQ ID NO: 2);

(SEQ ID NO: 1) and (SEQ ID NO: 17);

(SEQ ID NO: 1) and (SEQ ID NO: 19);

(SEQ ID NO: 5) and (SEQ ID NO: 6);

(SEQ ID NO: 9) and (SEQ ID NO: 10);

(SEQ ID NO: 13) and (SEQ ID NO: 14);

(SEQ ID NO: 18) and (SEQ ID NO: 17); and

(SEQ ID NO:18) and (SEQ ID NO: 19),

wherein, optionally, the set of primers further comprises at least one additional primer pair other than the at least one primer pair.

In one aspect, the present invention provides an allele-specific primer pair comprising:

a) a first primer comprising a first primer nucleotide sequence selected from the group consisting of (SEQ ID NO: 1), (SEQ ID NO: 2), (SEQ ID NO: 5), (SEQ ID NO: 6), (SEQ ID NO: 9), (SEQ ID NO: 10), (SEQ ID NO: 13), (SEQ ID NO: 14), (SEQ ID NO: 17), (SEQ ID NO: 18) and (SEQ ID NO: 19), wherein the first primer has a total nucleotide length of about 18 to about 50 nucleotides; and

b) a second primer comprising a second primer nucleotide sequence or complement thereof, wherein the second primer nucleotide sequence is selected from the group consisting of (SEQ ID NO: 3), (SEQ ID NO: 4), (SEQ ID NO: 7), (SEQ ID NO: 8), (SEQ ID NO: 11), (SEQ ID NO: 12), (SEQ ID NO: 15), (SEQ ID NO: 16), (SEQ ID NO: 20) and (SEQ ID NO: 21), wherein the second primer has a total nucleotide length of about 18 to about 50 nucleotides.

In another aspect, the present invention provides an isolated polynucleotide conjugated to a detectable label, wherein the polynucleotide comprises a nucleotide sequence or complement thereof, wherein the nucleotide sequence is selected from the group consisting of (SEQ ID NO: 1), (SEQ ID NO: 2), (SEQ ID NO: 3), (SEQ ID NO: 4), (SEQ ID NO: 5), (SEQ ID NO: 6), (SEQ ID NO: 7), (SEQ ID NO: 8), (SEQ ID NO: 9), (SEQ ID NO: 10), (SEQ ID NO: 11), (SEQ ID NO: 12), (SEQ ID NO: 13), (SEQ ID NO: 14), (SEQ ID NO: 15), (SEQ ID NO: 16), (SEQ ID NO: 17), (SEQ ID NO: 18), (SEQ ID NO: 19), (SEQ ID NO: 20) and (SEQ ID NO: 21), wherein the isolated polynucleotide has a total nucleotide length of about 18 to about 50 nucleotides.

In other aspects, the present invention provides an isolated polynucleotide conjugated to a detectable label, wherein the polynucleotide consists essentially of a nucleotide sequence or complement thereof, wherein the nucleotide sequence is selected from the group consisting of (SEQ ID NO: 1), (SEQ ID NO: 2), (SEQ ID NO: 3), (SEQ ID NO: 4), (SEQ ID NO: 5), (SEQ ID NO: 6), (SEQ ID NO: 7), (SEQ ID NO: 8), (SEQ ID NO: 9), (SEQ ID NO: 10), (SEQ ID NO: 11), (SEQ ID NO: 12), (SEQ ID NO: 13), (SEQ ID NO: 14), (SEQ ID NO: 15), (SEQ ID NO: 16), (SEQ ID NO: 17), (SEQ ID NO: 18), (SEQ ID NO: 19), (SEQ ID NO: 20) and (SEQ ID NO: 21).

In some aspects, the present invention provides a method for determining a CYP2C9 genotype of a subject. The method comprises:

a) contacting a probe with a sample comprising a nucleic acid having a sequence corresponding to the CYP2C9 genotype of the subject, wherein the probe comprises at least one isolated polynucleotide comprising a sequence consisting of at least 10, at least 18, at least 20, or at least 50 contiguous nucleotides or a complement thereof, wherein the contiguous nucleotides are contained in a nucleotide sequence selected from the group consisting of: (SEQ ID NO: 3), (SEQ ID NO: 4), (SEQ ID NO: 7), (SEQ ID NO: 8), (SEQ ID NO: 20), and (SEQ ID NO: 21); and

b) determining the CYP2C9 genotype of the subject, wherein selective hybridization of the probe to the nucleic acid is indicative of the CYP2C9 genotype.

In one aspect, the present invention provides a method for determining a VKORC1 genotype of a subject. The method comprises:

a) contacting a probe with a sample comprising a nucleic acid having a sequence corresponding to the VKORC1 genotype of the subject, wherein the probe comprises at least one isolated polynucleotide comprising a nucleotide sequence or complement thereof, wherein the nucleotide sequence is selected from the group consisting of (SEQ ID NO: 11), (SEQ ID NO: 12), (SEQ ID NO: 15), and (SEQ ID NO: 16), wherein the isolated polynucleotide has a total nucleotide length of about 18 to about 50 nucleotides;

b) determining the VKORC1 genotype of the subject, wherein selective hybridization of the probe to the nucleic acid is indicative of the VKORC1 genotype.

In other aspects, the present invention provides a method for selecting a medication or an optimal dose of a medication for a subject. The method comprises:

a) genotyping CYP2C9, VKORC1, or both to determine a genotype and, optionally, genotyping at least one additional gene; and

b) selecting the medication or the optimal dose of the medication based on the genotyping of step a).

In some aspects, the present invention provides a method for selecting an optimal dose of a medication for a human subject, the method comprising:

a) genotyping CYP2C9, VKORC1, or both to determine a genotype and, optionally, genotyping at least one additional gene; and

b) selecting the optimal dose of the medication based on the genotyping of step a), wherein the selecting further comprises using an algorithm based on the subject's CYP2C9 and/or VKORC1 genetic polymorphism, and one or more characteristics of the subject.

In one aspect, the present invention provides a method for determining an optimal dose of warfarin for a human subject. The method comprises:

a) genotyping CYP2C9, VKORC1, or both to determine a genotype and, optionally, genotyping at least one additional gene; and

b) selecting the optimal dose of warfarin based on the genotyping of step a), wherein the selecting further comprises using an algorithm based on the subject's CYP2C9 and/or VKORC1 genetic polymorphism, and one or more characteristics of the subject.

In a further aspect, the present invention provides an article of manufacture comprising one or more of the isolated polynucleotides described herein.

### BRIEF DESCRIPTION OF THE DRAWINGS

FIG. 1 shows partial nucleotide sequence corresponding to human CYP2C9 gene as provided by GenBank Accession Number AY341248, showing alignment of sequences of the CYP2C9-specific polypeptides (i.e., primers and probes) of the present invention. A number of features are shown for one embodiment of the present invention, including the following: probe sequence (shaded); polymorphic site (double underline); and primer sequence (underline).

FIG. 2 shows partial nucleotide sequence corresponding to human VKORC1 gene as provided by GenBank Accession Number AY587020, showing alignment of sequences of the VKORC1-specific polypeptides (i.e., primers and probes) of the present invention. A number of features are shown for one embodiment of the present invention, including the following: probe sequence (shaded); polymorphic site (double underline); and primer sequence (underline).

FIG. 3(a-d) shows the real-time multiplex PCR amplification plots for each of the representative CYP2C9 and VKORC1 alleles (i.e. genotype) and the corresponding data based on DNA sequencing.

FIG. 4 shows a confidence interval plot of delta Ct values for various genotypes using DNA extracted from blood or saliva. The confidence interval for each interval is 95%.

FIG. 5 shows real-time PCR amplification plots obtained for CYP2C9 alleles using ABI 7500 instrument to show cross-platform compatibility of the polynucleotide primers and probes of the present invention.

FIG. 6 shows real-time PCR amplification plots obtained for VKORC1 alleles using ABI 7500 instrument to show cross-platform compatibility of the polynucleotide primers and probes of the present invention.

FIG. 7 shows shows real-time PCR amplification plots obtained for CYP2C9 alleles using Stratagene Mx3005P instrument to show cross-platform compatibility of the polynucleotide primers and probes of the present invention.

FIG. 8 shows shows real-time PCR amplification plots obtained for VKORC1 alleles using Stratagene Mx3005P instrument to show cross-platform compatibility of the polynucleotide primers and probes of the present invention.

### DETAILED DESCRIPTION

With reference to the nomenclature derived from Accession Number AY341248, alleles of CYP2C9 include i) C and A at nucleotide positions 6312 and 45324, respectively (e.g., CYP2C9*1; a.k.a wild-type (Arg144 and Ile359)); ii) T at position 6312 (e.g., CY2C9*2 (a.k.a Cys144)); and C at position 45324 (e.g., CYP2C9*3 (a.k.a wild-type (Leu359)). Haining et al., 1996, Arch Biochem Biophys, 333:447-458; Hruska et al., Clinical Chemistry, 2004, 50: 2392-2395; and Miners et al., 1998, Br. J. Clin. Pharmacol, 45:525-538. And, with reference to the nomenclature derived from Accession Number AY587020, alleles of VKORC1 include i) C at position 1173 in intron 1 of VKORC1 (a.k.a wild-type), ii) T at position 1173 in intron 1 (a.k.a 1173 C>T variation), iii) G at position 3730 in the 3′ untranslated region (a.k.a wild-type), and iv) A at position 3730 in the 3′ untranslated region (a.k.a. 3730 G>A variation).

Without being held to a particular theory, it is believed that the CYP2C9 and VKORC1 genes encode products that influence the metabolism of a medication or that are associated with a treatment response to a medication (e.g., dosing). Accordingly, the present invention provides compositions and methods for selecting a medication or an optimal dose of a medication for a subject based on the subject's genotype for CYP2C9 and VKORC1. The term “subject” herein refers to a human, a eukaryote, an animal, a vertebrate animal, a mammal, a rodent (e.g., a guinea pig, a hamster, a rat, a mouse), murine (e.g., a mouse), canine (e.g., a dog), feline (e.g., a cat), equine (e.g., a horse), a primate, simian (e.g., a monkey or ape), a monkey (e.g., marmoset, baboon), or an ape (e.g., gorilla, chimpanzee, orangutang, gibbon).

### I. Polynucleotides

The term “nucleoside” refers to a base linked to a sugar. The base may be adenine (A), guanine (G) (or its substitute, inosine (I)), cytosine (C), or thymine (T) (or its substitute, uracil (U)). The sugar may be ribose (the sugar of a natural nucleotide in RNA) or 2-deoxyribose (the sugar of a natural nucleotide in DNA).

As used herein, unless expressly noted otherwise, the term “nucleoside triphosphate” or reference to any specific nucleoside triphosphate; e.g., adenosine triphosphate, guanosine triphosphate or cytidine triphosphate, refers to a triphosphate comprising either a ribonucleoside or a 2′-deoxyribonucleoside.

A “nucleotide” refers to a nucleoside linked to a single phosphate group.

A “natural nucleotide” refers to an A, C, G or U nucleotide when referring to RNA and to dA, dC, dG and dT (the “d” referring to the fact that the sugar is a deoxyribose) when referring to DNA. A natural nucleotide also refers to a nucleotide which may have a different structure from the above, but which is naturally incorporated into a polynucleotide sequence by the organism which is the source of the polynucleotide.

As used herein, a “modified nucleotide” refers to a “non-natural” nucleotide. A “non-natural” nucleotide may be a natural nucleotide that is placed in non-natural surroundings. For example, in a polynucleotide that is naturally composed of deoxyribonucleotides, e.g., DNA, a ribonucleotide would constitute a “non-natural” nucleotide. Similarly, in a polynucleotide that is naturally composed of ribonucleotides, i.e., RNA, a deoxyribonucleotide would constitute a non-natural nucleotide. A “non-natural” nucleotide also refers to a natural nucleotide that has been chemically altered. For example, without limitation, one or more substituent groups may be added to the base, sugar, or phosphate moieties of the nucleotide. On the other hand, one or more substituents may be deleted from the base, sugar or phosphate moiety. Or, one or more atoms or substituents may be substituted for one or more others in the nucleotide. A “modified” nucleotide may also be a molecule that resembles a natural nucleotide little, if at all, but is nevertheless capable of being incorporated by a polymerase into a polynucleotide in place of a natural nucleotide. The modified nucleotide may be a base-modified nucleotide. By “base-modified nucleotide” is meant a nucleotide in which the normal heterocyclic nitrogen base, adenine, guanine, cytosine, thymine, or uracil, is chemically modified by the addition, deletion and/or substitution of one or more substituents or atoms for that in the normal base.

The term “polynucleotide” refers to primers, probes, oligomer fragments to be detected, labeled-replication blocking probes, oligomer controls, and shall be generic to polydeoxyribonucleotides (containing 2-deoxy-D-ribose), to polyribonucleotides (containing D-ribose) and to any polynucleotide which is an N-glycoside of a purine or pyrimidine base (nucleotide), or modified purine or pyrimidine base. Also included in the definition of “polynucleotide” are nucleic acid analogs (e.g., peptide nucleic acids) and those that have been structurally modified (e.g., phosphorothioate linkages). Thus, the term “polynucleotide” includes a nucleic acid comprising one or more natural and/or modified nucleotide residues.

A “sequence” or “nucleotide sequence” refers to the order of nucleotide residues in a polynucleotide. There is no intended distinction between the length of a “nucleic acid”, “polynucleotide” or an “oligonucleotide”.

A “primer” or “probe” refers to a polynucleotide (synthetic or occurring naturally) comprising a nucleotide sequence that is complementary to a nucleotide sequence present in a nucleic acid molecule of interest and can form a duplexed structure by hybridization with the target sequence. Typically, gene specific polynucleotide probes comprising contiguous nucleotides may be used in sequence-dependent methods of gene identification. For example, probes may be labeled, e.g. with an energy transfer pair comprised of a fluorescent reporter and quencher. A polynucleotide that is used as a “probe” can also be adapted to function as a primer.

A “primer” is capable of acting as a point of initiation of nucleic acid synthesis or replication along a complementary strand when placed under conditions in which synthesis of a complementary strand is catalyzed by a polymerase. A “prime” that is capable of undergoing primer extension for the polymerization of nucleotides may also be utilized by the skilled artisan to function as a probe.

A “primer pair” comprises one primer that is complementary to a nucleic acid sequence present on the sense strand of a nucleic acid of interest and another primer that is complementary to a nucleic acid sequence present on the antisense strand of the nucleic acid of interest A “primer pair” can be used to amplify a specific region of the nucleic acid of interest by the process of forming extension products involving extending the annealed primers from a 3′ terminus of each primer to synthesize an extension product that is complementary to the target nucleic acid strands annealed to each primer wherein each extension product after separation from the target nucleic acid serves as a template for the synthesis of an extension product for the other primer of each pair (e.g., PCR amplification). The amplified products can be detected by a variety of methods known to the skilled artisan. U.S. Pat. No. 4,683,195 (Mullis et al.) describes a process for amplifying nucleic acid.

A “highly stringent condition,” as defined herein with respect to nucleic acid hybridization, can be identified by a condition that comprises the use of relatively low ionic strength solutions and high temperatures for washing. For example, a “highly” stringent condition can be identified by hybridization at 42° C. in 2×SSC (0.3M NaCl/0.03 M sodium citrate/0.1% sodium dodecyl sulfate (SDS)) and washing in 0.1×SSC (0.015M NaCl/0.0015 M sodium citrate), 0.1% SDS at 65° C.).

A “moderately stringent condition,” as defined herein with respect to nucleic acid hybridization, can be identified by washing and/or hybridization conditions less stringent than those described above for a highly stringent condition. An example of a moderately stringent condition is overnight incubation at 37° C. in a solution comprising: 20% formamide, 5×SSC (150 mM NaCl, 15 mM trisodium citrate), 50 mM sodium phosphate (pH 7.6), 5× Denhardt's solution, 10% dextran sulfate, and 20 mg/ml denatured sheared salmon sperm DNA, followed by washing in 1×SSC at about 35-50° C.

Stringency conditions relate to the set of conditions under which nucleic acid hybrids comprising double-stranded regions are formed and/or maintained. It is well known in the art that two complementary single-stranded nucleic acids (DNA or RNA) can anneal to one another so that complexes called hybrids are formed. Formation or subsequent stability of a formed hybrid can be affected by the conditions under which hybridization (i.e., annealing) occurs, by any wash conditions subsequent to hybridization, or both. Thus, through one or more nucleic acid hybridization steps, which can precede one or more wash steps, two nucleic acid sequences having a certain degree of complementary identity to one another can anneal together and form a hybrid comprising one or more contiguous regions of double-stranded nucleic acid. Further, formation of hybrids can occur in a variety of environments such as, for example, in solution, with one component immobilized on a solid support such as a nylon membrane, nitrocellulose paper, polystyrene, or in situ (e.g., in suitably prepared cells or histological sections).

It is well known in the art that a number of factors affect hybrid formation and/or stability such as, for example, temperature, duration, frequency, or salt or detergent concentration of the hybridization and/or wash conditions. Thus, for example, the stringency of a condition can be primarily due to the wash conditions, particularly if the hybridization condition used is one which allows less stable hybrids to form along with stable hybrids (e.g., wash conditions at higher stringency can remove less stable hybrids). In general, longer sequences require higher temperatures for proper annealing, while shorter sequences need lower temperatures. Hybridization generally depends on the ability of denatured nucleic acids to reanneal when complementary strands are present in a favorable environment at temperatures below their melting temperature. The higher the degree of desired homology between two sequences, the higher the relative temperature which can be used. As a result, it follows that higher relative temperatures would tend to make the reaction conditions more stringent, while lower temperatures less so.

Generally, stringency can be altered or controlled by, for example, manipulating temperature and salt concentration during hybridization and washing. For example, a combination of high temperature and low salt concentration increases stringency. The skilled artisan will recognize how to adjust the temperature, ionic strength, etc. of the stringent condition as necessary to accommodate factors such as polynucleotide length and the like.

Sometimes, nucleic acid duplex or hybrid stability is expressed as the melting temperature or Tₘ, which is the temperature at which 50% of one nucleic acid dissociates from a nucleic acid duplex. Accordingly, this melting temperature can be used to define the required stringency conditions. If sequences are related and substantially identical to each other, rather than identical, then it can be useful to first establish the lowest temperature at which only homologous annealing occurs with a particular concentration of salt (e.g., SSC or SSPE). Then, assuming 1% mismatching results in a 1° C. decrease in the Tₘ, the temperature of the final wash in the hybridization reaction is reduced accordingly (for example, if sequences having >95% identity with each other are sought, the final wash temperature is decreased by 5° C.). In practice, the change in Tₘ can be between 0.5° C. and 1.5° C. per 1% mismatch.

The term “consisting essentially of” means that immediately adjacent at their 5′ and/or 3′ end, the nucleotide sequences described herein do not have any further nucleotide sequences which are present in the CYP2C9 or VKORC1 gene. In other words, other nucleotide sequences derived from the CYP2C9 or VKORC1 gene may be present in the polynucleotides of the present invention provided they are not immediately adjacent at the 5′ and/or 3′ end of the nucleotide sequence. Thus, the term “consisting essentially of” is not intended to exclude a polynucleotide having a primer and probe linked to each other (e.g., Scorpion primer/probe) so that the binding of the probe to the amplicon is a unimolecular reaction. However, other moieties such as labels as well as nucleotide sequences which are not present in the corresponding gene may be present in the polynucleotides of the invention including being present immediately adjacent at the 5′ and/or 3′ end of the nucleotide sequence.

In one aspect, the present invention provides nucleic acid molecules that are useful for detection of polymorphisms (e.g., single nucleotide polymorphism (SNP)) in the CYP2C9 or VKORC1 gene. Accordingly, the invention includes an isolated polynucleotide comprising a nucleotide sequence or complement thereof of the sequences represented by the following:

(SEQ ID NO: 1)
5′-CCCCTGAATTGCTACAACAAATGTG-3′;
(SEQ ID NO: 2)
5′-GACTTCGAAAACATGGAGTGCA-3′;
(SEQ ID NO: 3)
5′-CAGAGATACCTTGACCTTC-3′;
(SEQ ID NO: 4)
5′-CCAGAGATACATTGACCTTC-3′;
(SEQ ID NO: 5)
5′-CTCATGACGCTGCGGAATTT-3′;
(SEQ ID NO: 6)
5′-GAAGATAGTAGTCCAGTAAGGTCAGTGATATG-3′;
(SEQ ID NO: 7)
5′-CATTGAGGACCGTGTTCA-3′;
(SEQ ID NO: 8)
5′-CATTGAGGACTGTGTTCAA-3′;
(SEQ ID NO: 9)
5′-ATCCTGACGTGGCCAAAGG-3′;
(SEQ ID NO: 10)
5′-CCACCTGGGCTATCCTCTGTT-3′;
(SEQ ID NO: 11)
5′-CCAGGAGATCATCGACCCTTGGAC-3′;
(SEQ ID NO: 12)
5′-CCAGGAGATCATCGACTCTTGGACTAGG-3′;
(SEQ ID NO: 13)
5′-CCCTAGATGTGGGGCTTCTAGATTA-3′;
(SEQ ID NO: 14)
5′-AGCGTGTGGCACATTTGGT-3′;
(SEQ ID NO: 15)
5′-CTCCTGCCATACCCACACATGACAAT-3′;
(SEQ ID NO: 16)
5′-CTCCTGCCATACCCGCACATGA-3′;
(SEQ ID NO: 17)
5′-GACTTCGAAAACATGGAGTTGCA-3′;
(SEQ ID NO: 18)
5′-TGCAAGACAGGAGCCACATG-3′;
(SEQ ID NO: 19)
5′-TTACCTTGGGAATGAGATAGTTTCTG-3′;
(SEQ ID NO: 20)
5′-TCCAGAGATACCTTGACCTTCTC-3′;
and
(SEQ ID NO: 21)
5′-TCCAGAGATACATTGACCTTCTC-3′,

wherein the isolated polynucleotide has a total nucleotide length of about 18 to about 50 nucleotides.

In another aspect, the present invention provides nucleic acid molecules that are useful for detection of polymorphisms (e.g., single nucleotide polymorphism (SNP)) in the CYP2C9 gene. Accordingly, the invention includes an isolated polynucleotide comprising a sequence consisting of at least 10, at least 18, at least 20, or at least 50 contiguous nucleotides or a complement thereof of the sequences represented by the following:

(SEQ ID NO: 1)
5′-CCCCTGAATTGCTACAACAAATGTG-3′;
(SEQ ID NO: 2)
5′-GACTTCGAAAACATGGAGTGCA-3′;
(SEQ ID NO: 3)
5′-CAGAGATACCTTGACCTTC-3′;
(SEQ ID NO: 4)
5′-CCAGAGATACATTGACCTTC-3′;
(SEQ ID NO: 5)
5′-CTCATGACGCTGCGGAATTT-3′;
(SEQ ID NO: 6)
5′-GAAGATAGTAGTCCAGTAAGGTCAGTGATATG-3′;
(SEQ ID NO: 7)
5′-CATTGAGGACCGTGTTCA-3′;
(SEQ ID NO: 8)
5′-CATTGAGGACTGTGTTCAA-3′;
(SEQ ID NO: 17)
5′-GACTTCGAAAACATGGAGTTGCA-3′;
(SEQ ID NO: 18)
5′-TGCAAGACAGGAGCCACATG-3′;
(SEQ ID NO: 19)
5′-TTACCTTGGGAATGAGATAGTTTCTG-3′;
(SEQ ID NO: 20)
5′-TCCAGAGATACCTTGACCTTCTC-3′;
and
(SEQ ID NO: 21)
5′-TCCAGAGATACATTGACCTTCTC-3′.

In one embodiment, the invention includes an isolated polynucleotide consisting essentially of a nucleotide sequence or complement thereof of the sequences represented by the following: (SEQ ID NO: 1), (SEQ ID NO: 2), (SEQ ID NO: 3), (SEQ ID NO: 4), (SEQ ID NO: 5), (SEQ ID NO: 6), (SEQ ID NO: 7), (SEQ ID NO: 8), (SEQ ID NO: 17), (SEQ ID NO: 18), (SEQ ID NO: 19), (SEQ ID NO: 20). and (SEQ ID NO: 21).

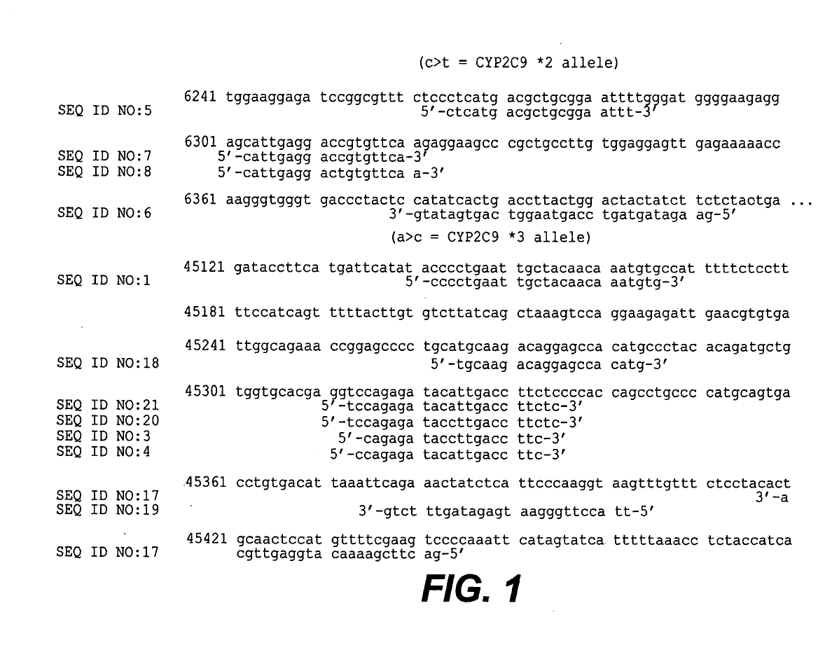

The nucleotide sequence alignment in FIG. 1 illustrates the position of SEQ ID NOs: 1, 3-8, and 17-21 relative to the CYP2C9 gene sequence derived from Accession Number AY341248.

In other aspects, the present invention provides nucleic acid molecules that are useful for detection of polymorphisms (e.g., single nucleotide polymorphism (SNP)) in the VKORC1 gene. Accordingly, the invention includes an isolated polynucleotide comprising a sequence consisting of at least 10, at least 18, at least 20, or at least 50 contiguous nucleotides or a complement thereof of the sequences represented by the following:

| | |
|---|---|
| 5′-ATCCTGACGTGGCCAAAGG-3′; | (SEQ ID NO: 9) |
| 5′-CCACCTGGGCTATCCTCTGTT-3′; | (SEQ ID NO: 10) |
| 5′-CCAGGAGATCATCGACCCTTGGAC -3′; | (SEQ ID NO: 11) |
| 5′-CCAGGAGATCATCGACTCTTGGACTAGG-3′; | (SEQ ID NO: 12) |
| 5′-CCCTAGATGTGGGGCTTCTAGATTA-3′; | (SEQ ID NO: 13) |
| 5′-AGCGGTGGCACATTTGGT-3′; | (SEQ ID NO: 14) |
| 5′-CTCCTGCCATACCCACACATGACAAT-3′; | (SEQ ID NO: 15) |
| and | |
| 5′-CTCCTGCCATACCCGCACATGA-3′. | (SEQID NO: 16) |

In another embodiment, the invention includes an isolated polynucleotide consisting essentially of a nucleotide sequence or complement thereof of the sequences represented by the following: (SEQ ID NO: 9), (SEQ ID NO: 10), (SEQ ID NO: 11), (SEQ ID NO: 12), (SEQ ID NO: 13), (SEQ ID NO: 14), (SEQ ID NO: 15), and (SEQ ID NO: 16).

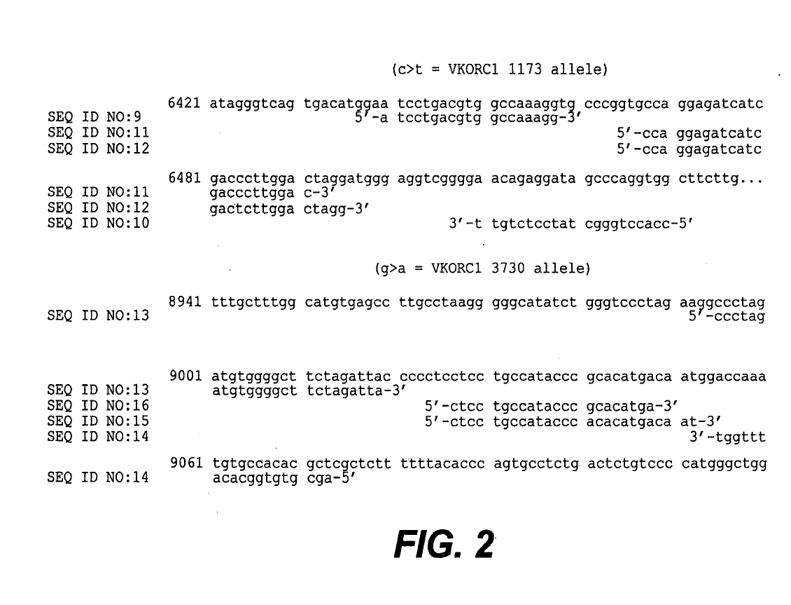

The nucleotide sequence alignment in FIG. 2 illustrates the position of SEQ ID NOS: 9-16 relative to the VKORC1 gene sequence derived from Accession Number AY587020.

In one aspect, the present invention provides nucleic acid molecules that comprise a polymorphic nucleotide residue (e.g., a single nucleotide polymorphism (SNP)) of the CYP2C9 or VKORC1 gene. Accordingly, the present invention provides an isolated polynucleotide comprising a sequence consisting of at least 10, at least 18, at least 20, or at least 50 contiguous nucleotides or a complement thereof of the sequences represented by the following: (SEQ ID NO: 3), (SEQ ID NO: 4), (SEQ ID NO: 7), (SEQ ID NO: 8), (SEQ ID NO: 11), (SEQ ID NO: 12), (SEQ ID NO: 15), (SEQ ID NO: 16), (SEQ ID NO: 20) and (SEQ ID NO: 21).

Each individual polynucleotide described herein, or a complement thereof, may be adapted to serve as a primer, either singly or in combination with at least one other primer. For example, a primer pair may be used for amplification of a specific region of the CYP2C9 or VKORC1 gene. Accordingly, in one embodiment, the present invention provides a primer pair comprising a first primer and a second primer each having a sequence consisting of at least 10, at least 18, at least 20, or at least 50 contiguous nucleotides or a complement thereof of the sequences represented by the following primer pairs:

(SEQ ID NO: 1) and (SEQ ID NO: 2);

(SEQ ID NO: 1) and (SEQ ID NO: 17);

(SEQ ID NO: 1) and (SEQ ID NO: 19);

(SEQ ID NO: 5) and (SEQ ID NO: 6);

(SEQ ID NO: 9) and (SEQ ID NO: 10);

(SEQ ID NO: 13) and (SEQ ID NO: 14);

(SEQ ID NO: 18) and (SEQ ID NO: 17); and

(SEQ ID NO:18) and (SEQ ID NO: 19).

A primer pair comprising a first primer and a second primer may be used to amplify CYP2C9 gene sequences to produce an amplified molecule (i.e., an amplicon). More specifically, a primer pair comprising a first primer and a second primer each having a sequence consisting of at least 10, at least 18, at least 20, or at least 50 contiguous nucleotides or a complement thereof of the sequences represented by the following primer pairs:

(SEQ ID NO: 1) and (SEQ ID NO: 2);

(SEQ ID NO: 1) and (SEQ ID NO: 17);

(SEQ ID NO: 1) and (SEQ ID NO: 19);

(SEQ ID NO: 18) and (SEQ ED NO: 17); and

(SEQ ID NO:18) and (SEQ ID NO: 19),

may be used to amplify the region of the CYP2C9 gene that is associated with the allelic variant CYP2C9*3 (Ile359Leu); and wherein the sequences are represented by the following primer pair (SEQ ID NO: 5) and (SEQ ID NO: 6), the primer pair may be used to amplify the region of the CYP2C9 gene that is associated with the allelic variant CYP2C9*2 (Arg144Cys).

Also, a primer pair comprising a first primer and a second primer each having a sequence consisting of at least 10, at least 18, at least 20, or at least 50 contiguous nucleotides or a complement thereof of the sequences represented by the following primer pairs:

(SEQ ID NO: 9) and (SEQ ID NO: 10); and

(SEQ ID NO: 13) and (SEQ ID NO: 14),

may be used to amplify VKORC1 gene sequences. More specifically, wherein the sequences are represented by the following primer pairs: (SEQ ID NO: 9) and (SEQ ID NO: 10), the primer pair may be used to amplify the region of the VKORC1 gene that is associated with the 1173 C>T variation; and wherein the sequences are represented by the following primer pairs: (SEQ ID NO: 13) and (SEQ ID NO: 14), the primer pair may be used to amplify the region of the VKORC1 gene that is associated with the 3730 G>A variation.

In another aspect, the present invention provides primers that may be used to amplify a product only when a specific allelic variant is present i.e., allele-specific primer. Accordingly, in one embodiment, the present invention includes an allele-specific primer pair comprising:

a) a first primer having a sequence consisting of at least 10, at least 18, at least 20, or at least 50 contiguous nucleotides or a complement thereof of the sequences represented by the following: (SEQ ID NO: 1), (SEQ ID NO: 2), (SEQ ID NO: 5), (SEQ ID NO: 6), (SEQ ID NO: 9), (SEQ ID NO: 10), (SEQ ID NO: 13), (SEQ ID NO: 14), (SEQ ID NO: 17), (SEQ ID NO: 18) and (SEQ ID NO: 19); and

b) a second primer having a sequence consisting of at least 10, at least 18, at least 20, or at least 50 contiguous nucleotides or a complement thereof of the sequences represented by the following: (SEQ ID NO: 3), (SEQ ID NO: 4), (SEQ ID NO: 7), (SEQ ID NO: 8), (SEQ ID NO: 11), (SEQ ID NO: 12), (SEQ ID NO: 15), (SEQ ID NO: 16), (SEQ ID NO: 20) and (SEQ ID NO: 21).

In some embodiments, the primer has a total nucleotide length of at least about 10 nucleotides, illustratively, about 10 to about 50, about 20 to about 45, about 24 to about 40, about 26 to about 35, and about 30 to about 32 nucleotides.

Various other primers, or variations of the primers described herein, may also be prepared and used in accordance with the present invention. For example, alternative primers can be designed based on targeted regions of the CYP2C9 and VKORC1 gene known or suspected to contain regions possessing high G/C content (i.e., the percentage of guanine and cytosine residues). As used herein, a “high G/C content” in a target nucleic acid, typically includes regions having a percentage of guanine and cytosine residues of about 60% to about 90%. Thus, changes in a prepared primer will alter, for example, the hybridization or annealing temperatures of the primer, the size of the primer employed, and the sequence of the specific amplification product. Therefore, manipulation of the G/C content, e.g., increasing or decreasing, of a primer or primer pair may be beneficial in increasing detection sensitivity in the method.

The polynucleotides (i.e., the primers and probes) described herein may be used in various combinations with each other or with other primers and probes that are specific to alleles other than those described herein.

### II. Labeled Polynucleotides

The term “label” as used herein refers to any constituent which can be used to provide a detectable (preferably quantifiable) signal, and which can be attached to a nucleic acid.

The polynucleotides of the present invention may comprise a label. In one embodiment, the present invention provides an isolated polynucleotide conjugated to a detectable label, wherein the polynucleotide comprises a sequence consisting of at least 10, at least 18, at least 20, or at least 50 contiguous nucleotides or a complement thereof of the sequences represented by the following: (SEQ ID NO: 1), (SEQ ID NO: 2), (SEQ ID NO: 3), (SEQ ID NO: 4), (SEQ ID NO: 5), (SEQ ID NO: 6), (SEQ ID NO: 7), (SEQ ID NO: 8), (SEQ ID NO: 9), (SEQ ID NO: 10), (SEQ ID NO: 11), (SEQ ID NO: 12), (SEQ ID NO: 13), (SEQ ID NO: 14), (SEQ ID NO: 15), (SEQ ID NO: 16), (SEQ ID NO: 17), (SEQ ID NO: 18), (SEQ ID NO: 19), (SEQ ID NO: 20) and (SEQ ID NO: 21). In another embodiment, the sequences are represented by the following: (SEQ ID NO: 3), (SEQ ID NO: 4), (SEQ ID NO: 7), (SEQ ID NO: 8), (SEQ ID NO: 11), (SEQ ID NO: 12), (SEQ ID NO: 15), (SEQ ID NO: 16), (SEQ ID NO: 20) and (SEQ ID NO: 21).

In one embodiment, the present invention provides an isolated polynucleotide conjugated to a detectable label, wherein the polynucleotide consists essentially of a nucleotide sequence or complement thereof of the sequences represented by the following: (SEQ ID NO: 1), (SEQ ID NO: 2), (SEQ ID NO: 3), (SEQ ID NO: 4), (SEQ ID NO: 5), (SEQ ID NO: 6), (SEQ ID NO: 7), (SEQ ID NO: 8), (SEQ ID NO: 9), (SEQ ID NO: 10), (SEQ ID NO: 11), (SEQ ID NO: 12), (SEQ ID NO: 13), (SEQ ID NO: 14), (SEQ ID NO: 15), (SEQ ID NO: 16), (SEQ ID NO: 17), (SEQ ID NO: 18), (SEQ ID NO: 19), (SEQ ID NO: 20) and (SEQ ID NO: 21). In another embodiment, the sequences are represented by the following: (SEQ ID NO: 3), (SEQ ID NO: 4), (SEQ ID NO: 7), (SEQ ID NO: 8), (SEQ ID NO: 11), (SEQ ID NO: 12), (SEQ ID NO: 15), (SEQ ID NO: 16), (SEQ ID NO: 20) and (SEQ ID NO: 21).

Non-limiting examples of the label constituent include fluorophores, chromophores, quenchers, an isotopic label, a polypeptide label, or a dye release compound. The label constituent may be incorporated in the polynucleotide by including a nucleotide having the label attached thereto. Isotopic labels preferably include those compounds that are beta, gamma, or alpha emitters, more preferably isotopic labels are ³²P, ³⁵S, or ¹²⁵I. Suitable polypeptide labels that can be utilized in accordance with the present invention include antigens (e.g., biotin, digoxigenin, and the like) and enzymes (e.g., horse radish peroxidase). A dye release compound may include chemiluminescent systems defined as the emission of absorbed energy (typically as light) due to a chemical reaction of the components of the system, including oxyluminescence in which light is produced by chemical reactions involving oxygen.

One can also use both a fluorophore and quenching agent to label the probe. When the probe is intact, the fluorescence of the fluorophore is quenched by the quencher. Quenching involves transfer of energy between the fluorophore and the quencher, the emission spectrum of the fluorophore and the absorption spectrum of the quencher must overlap.

Any suitable fluorophore is included within the scope of the invention. Fluorophores that may be used in the methods of the present invention include, by way of example, 1,8-ANS, 4-methylumbelliferone, 5-carboxy-2,7-dichlorofluorescein, 5-carboxynapthofluorescein (pH 10), 5-FAM (5-carboxyfluorescein), 5-ROX (carboxy-X-rhodamine), 5-TAMRA (5-carboxytetramethylrhodamine, high pH>8), 6-Carboxyrhodamine 6G, 6-FAM, 7-AAD, 7-amino-4-methylcoumarin, 7-aminoactinomycinD (7-AAD), 7-hydroxy-4-methylcoumarin, ABQ, Acid Fuchsin, ACMA (9-amino-6-chloro-2-methoxyacridine), Acridine, Acridine Orange, Acridine Orange+DNA, Acridine Yellow, Alexa Fluor 350™, Alexa Fluor 488™, Alexa Fluor 532™, Alexa Fluor 546™, Alexa Fluor 555™, Alexa Fluor 568™, Alexa Fluor 594™, Alexa Fluor 647™, Alexa Fluor 660™, Alizarin Red, AMCA (Aminomethylcoumarin), AMCA-X, AmCyan, Aminoactinomycin D, Aminocoumarin, Anthrocyl stearate, APC (Allophycocyanin), Astrazon Brilliant Red 4G, Astrazon Orange R, Astrazon Yellow 7 GLL, Atabrine, ATTO-TAG™ CBQCA, Auramine, Aurophosphine, Beta lactamase, BFP (Blue Fluorescent Protein), Bisbenzimide (Hoechst), bis-BTC, Blancophor FFG, Blancophor SV, BOBO-1, BO-PRO-1, BOBO™-3, BODIPY, BODIPY 492/515, BODIPY 505/515, BODIPY 542/563, BODIPY 564/570, BODIPY 650/665 Dye, BODIPY 650/665-X, BODIPY FL-Br2, BODIPY TMR, BODIPY TR, BODIPY TR ATP, BODIPY TR-X dye, BODIPY TR-X SE, BO-PRO™-3, BTC, Calcein, Calcein Blue, Calcium Crimson™, Calcium Green-1, Calcium Green-2 (including Ca2+), Calcium Green-5N (including Ca2+), Calcium Orange, Calcofluor White, Carboxy SNARF Indicators, Cascade Blue™, Catecholamine, CFDA, CFP (cyan GFP), Chromomycin A, CI-NERF, CL-NERF, CMFDA, Coelenterazine F, Coumarin Phalloidin, CPM Methylcoumarin, Cy2™, Cy3™, Cy3.5™, Cy5™, Cyclic AMP Fluorosensor (FiCRhR), CyQuant Cell Proliferation Assay, DAPI, DCFDA, DCFH (Dichlorodihydrofluorescein Diacetate), DHR (Dihydrorhodamine 123), DiD (DiIC18(5))-Lipophilic Tracer, DiI, DiI (DiIC18(3)), DiO, DM-NERF, dsRed (Red Fluorescent Protein), DTAF, DY-635-NHS, EBFP, ECFP, Eosin, Ethidium Bromide, FAM, Fast Blue, FDA, Feulgen (Pararosanilin), FIF (Formaldehyde Induced Fluorescence), FITC (Fluorescein), Fluo-3, Fluo-4, Fluoro-Emerald, Fluor-Ruby, FluorX, Fura Red™ (high pH), Genacryl Brilliant Red B, Genacryl Brilliant Yellow 10GF, Genacryl Yellow 5GF, GFP (EGFP), Gloxalic Acid, Granular Blue, Haematoporphyrin, HcRed, HEX, Hoechst 33258, Hoechst 33342, Hoechst 34580, HPTS, Indo-1, Indodicarbocyanine (DiD), Intrawhite Cf, JC-1, JC-9, JOE, JO-JO-1, JO-PRO-1, Laurodan, Leucophor PAF, SF, WS, Lissamine Rhodamine, LIVE/DEAD Kit Animal Cells, LOLO-1, LO-PRO-1, LysoSensor Blue, LysoSensor Blue DND-167, Lysosensor Blue DND-192, LysoTracker Blue, LysoTracker Blue-White, LysoTracker Green, LysoTracker Red, LysoTracker Red DND-99 (L-7528), LysoTracker Yellow, Magdala Red (Phloxin B), Mag-Fura Red, Mag-Indo-1, Magnesium Green, Magnesium Orange, Marina Blue, Merocyanin, Methoxycoumarin, MitoTracker™ Green, MitoTracker™ Orange, MitoTracker™ Red, Mitramycin, Monochlorobimane, NBD, NBD Amine, NBD-X, NED, NeuroTrace 500/525 Green, NeuroTrace 500/525 Green Fluorescent Nissl Stain, Nile Red, Nile Red, Nissl, Nitrobenzoxadidole, Nylosan Brilliant Lavin EBG, Oregon Green™, Oregon Green™ 488, Oregon Green™ 500, Oregon Green™ 514, Pacific Blue™, PBFI, Phloxin B (Magdala Red), Phorwite AR, Phorwite BKL, Phorwite Rev, Phorwite RPA, Phycoerythrin (PE), PKH26, PKH26 (Sigma), PKH67, Pontochrome Blue Black, POPO-3, PO-PRO-3, Propidium Iodide (PI), Pyronin B, Resorufin, Rhod-123, Rhod-2, Rhodamine, Rhodamine 110, Rhodamine 123, Rhodamine 6G, Rhodamine B, Rhodamine B, Rhodamine B 200, Rhodamine BB, Rhodamine BG, Rhodamine Green, Rhodamine Phallicidine, Rhodamine Phalloidin, Rhodamine Red, Rhodamine Red Dye, Rhodol Green, Rose Bengal, ROX, R-phycoerythrin (PE), rsGFP (red shifted GFP, S65T), S65C, S65L, S65T, SBFI, Sevron Brilliant Red 2B, Sevron Brilliant Red B, Sevron Yellow L, sgBFP™, sgGFP™ (super glow GFP), SITS (Primuline), SITS (Stilbene Isothiosulphonic Acid), SNARF (carboxy) 488 Excitation pH6, SNARF (carboxy) 514 Excitation pH6, SNARF (carboxy) Excitation pH9, Sodium Green, SpectrumAqua, SpectrumRed, SpectrumGold, SpectrumGreen, SpectrumOrange, SPQ (6-methoxy-N-(3-sulfopropyl)quinolinium), Sulphorhodamine B can C, SYTO 64, SYTO Blue Fluorescent Nucleic Acid Stain 43, SYTO Blue Fluorescent Nucleic Acid Stain 44, SYTO Blue Fluorescent Nucleic Acid Stain 45, SYTO Green Fluorescent Nucleic Acid Stains 11, 14, 15, 20, 22, 25, SYTO Green Fluorescent Nucleic Acid Stains 12, 13, 16, 21, 23, 24, SYTO Orange Fluorescent Nucleic Acid Stains 80, 81, 82, 83, SYTO Orange Fluorescent Nucleic Acid Stains 84, 85, SYTO Red Fluorescent Nucleic Acid Stains 60, 62, 63, SYTO Red Fluorescent Nucleic Acid Stain 64, SYTOX Blue, SYTOX Green, TAMRA, TET, Tetramethylrhodamine, Rhodamine B, Texas Red”, Thiadicarbocyanine (DiSC3), Thiazine Red R, Thioflavin TCN, Thiolyte, Thiozole Orange, Tinopol CBS (Calcofluor White), TMR, TOTO-1, TO-PRO-1, TOTO-3, TO-PRO-3, TRITC (Tetramethylrhodamine, low pH<8), TRITC (Tetramethylrhodamine, high pH>8), True Blue, Ultralite, Uvitex SFC, wtGFP (wild type GFP, non-UV excitation), WW 781, X-Rhodamine, Xylene Orange, Y66H, Y66W, YFP (yellow GFP), YOYO-1, YO-PRO-1, derivatives of coumarin, etc.

Quenchers, for example, Dabcyl and TAMRA are well known quencher molecules that may be used in the methods of the present invention. However, the invention is not limited to the specific examples of fluorophores and quenchers disclosed herein.

In some embodiments, the polynucleotides of the present invention are labeled with the CAL Fluor, Quasar and BHQ dyes sold by Biosearch Technologies (Novato, Calif.). These 5′-fluorophores and 3′-quenchers can be incorporated into a variety of popular probe designs, including dual-labeled TaqMan® and Molecular Beacon probes and Black Hole Scorpions™ and Amplifluor® Direct primer systems. And, they are fully compatible with the range of real-time PCR instruments including the Applied Biosystems' and Stratagene real-time machines, the Corbett Rotor-Gene™ 6000, the Bio-Rad iQ5® and Cepheid SmartCycler®, among others.

In one embodiment, the polynucleotides of the present invention are labeled with a 5′-fluorophore/3′-quencher selected from Cal Red 610/BHQ-2, Quasar 670/BHQ-2, FAM/BHQ-1, Cal Orange 560/BHQ-1, or Cal Orange 560, BHQ-1.

Detecting a target nucleic acid (e.g., an amplicon) typically depends on a number of factors including the type of label and the genotyping method employed. For example, the polynucleotide labeled with the detectable label may be hybridized to a single-stranded target nucleic acid, after which the hybridized probe may be detected via the label. The label detection may be carried out by a method suitable for the particular label, and for example, when using an intercalator fluorescent dye for labeling the polynucleotide, a dye with the property of exhibiting increased fluorescent intensity by intercalation in the double-stranded nucleic acid comprising the target nucleic acid and the polynucleotide probe may be used in order to allow easy detection of only the hybridized probe without removal of the probe that has not hybridized to the target nucleic acid. When using a common fluorescent dye as the label, the label may be detected after removal of the probe that has not hybridized to the target nucleic acid. Alternatively, when incorporating the polynucleotide-labeled probe in the reaction solution during an amplification, it is especially preferable to modify the probe by, for example, adding glycolic acid to the 3′-end so that the probe will not function as a nucleotide primer. Other examples of labels are described herein in the context of various detection method.

### III. Genotyping

Genomic DNA may be used to determine genotype, although mRNA also can be used. Genomic DNA is typically extracted from a biological sample such as a peripheral blood sample, but can be extracted from other biological samples, including tissues (e.g., mucosal scrapings of the lining of the mouth or from renal or hepatic tissue), blood, saliva, and buccal cells. When saliva is analyzed, a sponge or saliva collection via buccal swab can be used to obtain the samples. This approach is much less invasive than taking blood samples, and the methods described herein are effective using such saliva samples.

Routine methods can be used to extract genomic DNA from a blood or tissue sample, including, for example, phenol extraction. Alternatively, genomic DNA can be extracted with kits such as the QIAamp® Tissue or Blood Kits (Qiagen, Valencia, Calif.), Wizard® Genomic DNA purification kit (Promega, Madison, Wis.), PUREGENE® DNA purification kit (Gentra Systems, Minneapolis, Minn.), Oragene™ DNA Self-Collection Kit (DNA Genotek Inc., Ontario, Canada), and the A.S.A.P.® Genomic DNA isolation kit (Boehringer Mannheim, Indianapolis, Ind.).

The polynucleotides described herein, such as primers and probes, may be used in methods to determine a CYP2C9 genotype of a subject. The method comprises:

a) contacting a probe with a sample comprising a nucleic acid having a sequence corresponding to the CYP2C9 genotype of the subject, wherein the probe comprises at least one isolated polynucleotide comprising a sequence consisting of at least 10, at least 18, at least 20, or at least 50 contiguous nucleotides or a complement thereof of the sequences represented by the following: (SEQ ID NO: 3), (SEQ ID NO: 4), (SEQ ID NO: 7), (SEQ ID NO: 8), (SEQ ID NO: 20), and (SEQ ID NO: 21); and

b) determining the CYP2C9 genotype of the subject, wherein selective hybridization of the probe to the nucleic acid is indicative of the CYP2C9 genotype.

In another embodiment, the CYP2C9 genotype is wild-type, CYP2C9*2, CYP2C9*3, or a combination thereof.

In some embodiments, the nucleic acid is an amplicon.

In other embodiments, the subject is a human.

In one embodiment, the method further comprises amplifying the nucleic acid having the sequence corresponding to the CYP2C9 genotype of the subject using at least one primer pair, wherein the at least one primer pair comprises a first primer and a second primer each having a sequence consisting of at least 10, at least 18, at least 20, or at least 50 contiguous nucleotides or a complement thereof of the sequences represented by the following primer pairs:

(SEQ ID NO: 1) and (SEQ ID NO: 2);

(SEQ ID NO: 1) and (SEQ ID NO: 17);

(SEQ ID NO: 1) and (SEQ ID NO: 19);

(SEQ ID NO: 5) and (SEQ ID NO: 6);

(SEQ ID NO: 18) and (SEQ ID NO: 17); and

(SEQ ID NO:18) and (SEQ ID NO: 19).

In another embodiment, amplifying comprises a polymerase chain reaction (PCR). In one aspect of this embodiment, the PCR is real-time PCR.

In one embodiment, the present invention provides a method for determining a VKORC1 genotype of a subject. The method comprises:

a) contacting a probe with a sample comprising a nucleic acid having a sequence corresponding to the VKORC1 genotype of the subject, wherein the probe comprises at least one isolated polynucleotide comprising a nucleotide sequence or complement thereof of the sequences represented by the following: (SEQ ID NO: 11), (SEQ ID NO: 12), (SEQ ID NO: 15), and (SEQ ID NO: 16), wherein the isolated polynucleotide has a total nucleotide length of about 18 to about 50 nucleotides;

b) determining the VKORC1 genotype of the subject, wherein selective hybridization of the probe to the nucleic acid is indicative of the VKORC1 genotype.

In another embodiment, the VKORC1 genotype is wild-type, 1173 C>T variation, 3730 G>A variation, or a combination thereof.

In some embodiments, the nucleic acid is an amplicon.

In other embodiments, the subject is a human.

In one embodiment, the method further comprises amplifying the nucleic acid having the sequence corresponding to the VKORC1 genotype of the subject using at least one primer pair, wherein the at least one primer pair comprises a first primer and a second primer each having a sequence consisting of at least 10, at least 18, at least 20, or at least 50 contiguous nucleotides or a complement thereof of the sequences represented by the following primer pairs:

(SEQ ID NO: 9) and (SEQ ID NO: 10); and

(SEQ ID NO: 13) and (SEQ ID NO: 14).

In another embodiment, amplifying comprises a polymerase chain reaction (PCR). In one embodiment, the PCR is real-time PCR.

In another embodiment, the probe is present in a composition comprising at least one isolated polynucleotide comprising a nucleotide sequence or complement thereof of the sequences represented by the following: (SEQ ID NO: 3), (SEQ ID NO: 4), (SEQ ID NO: 7), (SEQ ID NO: 8), (SEQ ID NO: 11), (SEQ ID NO: 12), and (SEQ ID NO: 15), (SEQ ID NO: 16), (SEQ ID NO: 20) and (SEQ ID NO: 21), wherein the isolated polynucleotide has a total nucleotide length of about 18 to about 50 nucleotides.

In one embodiment, the nucleic acid comprises a sequence corresponding to the CYP2C9 gene, the VKORC1 gene, or both. In another embodiment, the nucleic acid comprises the wild-type CYP2C9 gene, the CYP2C9 gene carrying the CYP2C9*2 polymorphism, the CYP2C9 gene carrying the CYP2C9*3 polymorphism, the wild-type VKORC1 gene, the VKORC1 gene carrying the 1173 C>T polymorphism, the VKORC1 gene carrying the 3730 G>A polymorphism, or any combination thereof. In some embodiments, the nucleic acid is genomic DNA.

### IV. Amplification

The polynucleotides of the present invention may be used in nucleic acid amplification methods including the techniques disclosed herein. Amplification techniques are well known in the art, and include methods such as real-time PCR, traditional PCR, nucleic acid sequence-based amplification (NASBA), and transcription mediated amplification (TMA).

General PCR techniques are described, for example in PCR Primer: A Laboratory Manual, Ed. by Dieffenbach, C. and Dveksler, G., Cold Spring Harbor Laboratory Press, 1995. When using RNA as a source of template, reverse transcriptase can be used to synthesize complementary DNA (cDNA) strands. Ligase chain reaction, strand displacement amplification, or self-sustained sequence replication also can be used to obtain isolated nucleic acids. See, e.g., Lewis, 1992, Genetic Engineering News 12:1; Guatelli et al., 1990, Proc. Natl. Acad. Sci. USA 87:1874-1878; and Weiss, 1991, Science 254:1292-1293.

Specific regions of mammalian DNA can be amplified (i.e., replicated such that multiple exact copies are produced) when a pair of polynucleotide primers is used in the same PCR reaction, wherein one primer contains a nucleotide sequence from the coding strand of a nucleic acid and the other primer contains a nucleotide sequence from the non-coding strand of the nucleic acid. The “coding strand” of a nucleic acid is the nontranscribed strand, which has the same nucleotide sequence as the specified RNA transcript (with the exception that the RNA transcript contains uracil in place of thymidine residues), while the “non-coding strand” of a nucleic acid is the strand that serves as the template for transcription.

A single PCR reaction mixture can include one pair of polynucleotide primers. Alternatively, a single reaction mixture can include a plurality of polynucleotide primer pairs, in which case multiple PCR products can be generated (e.g., 5, 10, 15, or 20 primer pairs). Each primer pair may be amplified, for example, one exon or a portion of one exon. Intron sequences also can be amplified. Exons or introns of a gene of interest also ma be amplified, then directly sequenced. Dye primer sequencing can be used to increase the accuracy of detecting heterozygous samples.

Allele-specific hybridization may be used to detect sequence variants (e.g., polymorphisms), including complete haplotypes of a mammal. See, Stoneking et al., 1991, Am. J. Hum. Genet. 48:370-382; and Prince et al., 2001, Genome Res., 11:152-162. For example, samples of DNA or RNA from one or more subjects may be amplified using pairs of primers and the resulting amplification products may be immobilized on a substrate (e.g., in discrete regions). Hybridization conditions are selected such that a nucleic acid probe can specifically bind to the sequence of interest, e.g., the variant nucleic acid sequence. Such hybridizations may be performed under high stringency as some sequence variants include only a single nucleotide difference (e.g., SNPs). A high stringency condition is as described above.

Hybridization conditions may be adjusted to account for unique features of the nucleic acid molecule, including length and sequence composition. Probes may be labeled (e.g., fluorescently) to facilitate detection. In some embodiments, one of the primers used in the amplification reaction is biotinylated (e.g., 5′ end of reverse primer) and the resulting biotinylated amplification product is immobilized on an avidin or streptavidin coated substrate (e.g., in discrete regions).

In one embodiment, real-time PCR can be used to determine genotype. A number of techniques for real-time detection of the products of an amplification reaction are known in the art. Many of these techniques produce a fluorescent read-out that may be continuously monitored (e.g., molecular beacons and fluorescent resonance energy transfer probes).

Real-time quantitation of PCR reactions may be accomplished using the TaqMan® system (Applied Biosystems). TaqMan®) probes are commercially available, and the TaqMan® system (Applied Biosystems) is well known in the art. TaqMan® probes anneal between the upstream and downstream primer in a PCR reaction. They contain a 5′-fluorophore and a 3′-quencher. During amplification the 5′-3′ exonuclease activity of the Taq polymerase cleaves the fluorophore off the probe. Since the fluorophore is no longer in close proximity to the quencher, the fluorophore will be allowed to fluoresce. The resulting fluorescence may be measured, and is in direct proportion to the amount of target sequence that is being amplified.

In the MGB Eclipse™ system (Nanogen Inc., Bothell, Wash.), wherein the MGB moiety is attached to the 3′-end or the 5′-end of a DNA probe during synthesis on a commercial synthesizer or post-synthetically to an amine modified oligo, the MGB moiety folds back into the minor groove formed by the DNA duplex to stabilize hybridization. The effect of this stabilization is an increase in melting temperature and the MGB moiety produces a “Tm leveling” effect as A-T content increases. The increase in the melting temperature due to the presence of the MGB moiety allows the use of shorter probes with improved mismatch discrimination.

In the Molecular Beacon system, the beacons are hairpin-shaped probes with an internally quenched fluorophore whose fluorescence is restored when bound to its target. The loop portion acts as the probe while the stem is formed by complimentary arm sequences at the ends of the beacon. A fluorophore and quenching moiety are attached at opposite ends, the stem keeping each of the moieties in close proximity, causing the fluorophore to be quenched by energy transfer. When the beacon detects its target, it undergoes a conformational change forcing the stem apart, thus separating the fluorophore and quencher. This causes the energy transfer to be disrupted to restore fluorescence.

A further real-time fluorescence based system which may be incorporated in the methods of the present invention is Zeneca's Scorpion system; and Whitcombe et al., (1999) Nature Biotechnology 17, 804-807, which are incorporated by reference in their entirety. The Scorpion method is based on a primer with a tail attached to its 5′ end by a linker that prevents copying of the 5′ extension. The probe element is designed so that it hybridizes to its target only when the target site has been incorporated into the same molecule by extension of the tailed primer.

Thus, in a further aspect of the present invention the products of nucleic acid amplification are detected using real-time techniques including, for example, real-time PCR. In one embodiment, the real-time technique comprises using the TaqMan® system, MGB eclipse, the Molecular beacons system, or the Scorpion probe system.

Nucleic acid sequence-based amplification (NASBA) is an isothermal transcription-based amplification method. The NASBA technology can be applied to SNP analysis using human genomic DNA as a template. Combination of DNA NASBA with multiplex hybridization of specific molecular beacons makes it possible to unambiguously discriminate the presence of the SNP of interest. This protocol makes it possible to rapidly detect single nucleotide substitutions in clinical or cell line DNA sequences using a large range of DNA input.

TMA (Gen-probe Inc., San Diego, Calif.) is an RNA transcription amplification system using two enzymes to drive the reaction, namely RNA polymerase and reverse transcriptase. The TMA reaction is isothermal and may amplify either DNA or RNA to produce RNA amplified end products. TMA may be combined with Gen-probe's Hybridization Protection Assay (HPA) detection technique to allow detection of products in a single tube.

Other methods of genotyping may be performed using various combinations of the polynucleotides of the present invention. For example, PCR conditions and primers can be developed that amplify a product only when a specific allelic variant is present i.e., allele-specific PCR). The subject's DNA may be amplified using primers specific for a particular allele and the amplification reactions examined for the presence of amplification products using standard methods to visualize the DNA. For example, samples containing solely the wild type allele would have amplification products only in the reaction using the wild type primer. Similarly, samples containing solely the variant allele would have amplification products only in the reaction using the variant primer. Allele-specific PCR also can be performed using allele-specific primers that introduce priming sites for two universal energy-transfer-labeled primers (e.g., one primer labeled with a green dye such as fluoroscein and one primer labeled with a red dye such as sulforhodamine). Amplification products can be analyzed for green and red fluorescence in a plate reader. See, Myakishev et al., 2001, Genome 11(1):163-169.

Mismatch cleavage methods also can be used to detect differing sequences by PCR amplification, followed by hybridization with the wild type sequence and cleavage at points of mismatch. Chemical reagents, such as carbodiimide or hydroxylamine and osmium tetroxide can be used to modify mismatched nucleotides to facilitate cleavage.

### IV. Pharmacogenomics

By utilizing pharmacogenomics, the present invention provides an effective method for selecting a medication or an optimal dose for a medication. Non-limiting examples of the medication include S-warfarin, R-warfarin, amitriptyline, caffeine, clomipramine, clozapine, cyclobenzaprine, estradiol, fluvoxamine, haloperidol, imipramine, mexilletine, naproxen, olanzapine, ondansetron, phenacetin, acetaminophen, propranolol, riluzole, ropivacaine, tacrine, theophylline, tizanidine, verapamil, zileuton, zolmitriptan bupropion, cyclophosphamide, efavirenz, ifosfamide, methadone, paclitaxel, torsemide, amodiaquine, cerivastatin, repaglinide, Proton Pump Inhibitors, lansoprazole, omeprazole, pantoprazole, rabeprazole, E-3810, diazepam, phenytoin(O), S-mephenytoin, phenobarbitone, amitriptyline, carisoprodol, citalopram, clomipramine, cyclophosphamide, hexobarbital, imipramine, indomethacin, R-mephobarbital, moclobemide, nelfinavir, nilutamide, primidone, progesterone, proguanil, propranolol, teniposide, diclofenac, ibuprofen, lomoxicam, meloxicam, S-naproxen, piroxicam, suprofen , tolbutamide, glipizide , losartan, irbesartan, glyburide, glibenclamide, glipizide, glimepiride, tolbutamide, amitriptyline, celecoxib, fluoxetine, fluvastatin glyburide, nateglinide, phenytoin, rosiglitazone, tamoxifen, torsemide, carvedilol, S-metoprolol, propafenone, timolol, amitriptyline, clomipramine, desipramine, imipramine, paroxetine, haloperidol, perphenazine, risperidone, thioridazine, zuclopenthixol, alprenolol, amphetamine, aripiprazole, atomoxetine, bufuralol, chlorpheniramine, chlorpromazine, codeine, debrisoquine, dexfenfluramine, dextromethorphan, duloxetine, encainide, flecainide, fluoxetine, fluvoxamine, lidocaine, metoclopramide, methoxyamphetamine, mexilletine, minaprine, nebivolol, nortriptyline, ondansetron, oxycodone, perhexiline, phenacetin, phenformin, promethazine, propranolol, sparteine, tamoxifen, tramadol, venlafaxine, enflurane, halothane, isoflurane, methoxyflurane, sevoflurane, acetaminophen, aniline, benzene, chlorzoxazone, ethanol, N,N-dimethyl formamide, theophylline, clarithromycin, erythromycin, telithromycin, quinidine, alprazolam, diazepam, midazolam, triazolam, cyclosporine, tacrolimus (FK506), indinavir, nelfinavir, ritonavir, saquinavir, cisapride, astemizole, chlorpheniramine, terfenidine, amlodipine, diltiazem, felodipine, lercanidipine, nifedipine, nisoldipine, nitrendipine, verapamil , atorvastatin, cerivastatin, lovastatin, simvastatin, estradiol, hydrocortisone, progesterone, testosterone, alfentanyl, aprepitant, aripiprazole, buspirone, cafergot, caffeine, cilostazol, cocaine, codeine-N-demethylation, dapsone, dexamethasone, dextromethorphan, docetaxel, domperidone, eplerenone, fentanyl, finasteride, gleevec, haloperidol, irinotecan, LAAM, lidocaine, methadone, nateglinide, odanestron, pimozide, propranolol, quetiapine, quinine, risperidone, salmeterol, sildenafil, sirolimus, tamoxifen, taxol, terfenadine, trazodone, vincristine, zaleplon, ziprasidone, and zolpidem.

In one embodiment, the medication is selected from an NSAID (e.g., diclofenac, ibuprofen, lomoxicam, meloxicam, S-naproxen, piroxicam, suprofen, an oral hypoglycemic agents (e.g., tolbutamide, glipizide), an angiotensin II blocker (e.g., losartan, irbesartan), or a sulfonylurea (e.g., glyburide, glibenclamide, glipizide, glimepiride, tolbutamide, amitriptyline, celecoxib, fluoxetine, fluvastatin glyburide, nateglinide, phenytoin, rosiglitazone, tamoxifen, torsemide, S-warfarin).

In another embodiment, the medication is the S-enantiomer of warfarin (i.e., S-warfarin).

Accordingly, in one embodiment, the present invention includes a method for selecting a medication or an optimal dose of a medication for a subject. The method comprises:

a) genotyping CYP2C9, VKORC1, or both to determine a genotype and, optionally, genotyping at least one additional gene; and

b) selecting the medication or the optimal dose of the medication based on the genotyping of step a).

The genotyping and the medication are as described above.

In some embodiments, the genotype comprises wild-type CYP2C9, CYP2C9*2, CYP2C9*3, wild-type VKORC1, VKORC1 1173 C>T, VKORC1 3730 G>A, or a combination thereof.

In addition to genotyping the subject's CYP2C9 and/or VKORC1, optionally, at least one additional gene may be genotyped. For example, the additional genes to be genotyped may include cytochrome P450 genes other than the gene that encodes CYP2C9. Or the additional genes may include genes that encode a product that relates to the ability of the subject to respond to a particular class of medication. For example, to select an antidepressant, the additional genes that may be genotyped may include a serotonin transporter gene and a serotonin receptor 2A gene.

Non-limiting examples of cytochrome P450 genes that may be genotyped are listed in Table 1 along with their respective polymorphisms.

TABLE 1
Cytochrome P450 Genes and Their Polymorphisms

| Gene | Allele | Polymorphism |
|---|---|---|
| 1A1 | *1A | Wild-type |
| | *2 | A2455G |
| | *3 | T3205C |
| | *4 | C2453A |
| 1A2 | *1A | Wild-type |
| | *1F | −164 C > A |
| | *3 | G1042A |
| 1B1 | *1 | Wild-type |
| | *2 | R48G |
| | *3 | L432V |
| | *4 | N453S |
| | *11 | V57C |
| | *14 | E281X |
| | *18 | G365W |
| | *19 | P379L |
| | *20 | E387K |
| | *25 | R469W |
| 2A6 | *1A | Wild-type |
| | *1B | CYP2A7 translocated to 3′-end |
| | *2 | T479A |
| | *5 | *1B + G6440T |
| 2B6 | *1 | Wild-type |
| | *2 | R22C |
| | *3 | S259C |
| | *4 | K262R |
| | *5 | R487C |
| | *6 | Q172H; K262R |
| | *7 | Q172H; K262R; R487C |
| 2C8 | *1A | Wild-type |
| | *1B | −271C > A |
| | *1C | −370T > G |
| | *2 | I269F |
| | *3 | R139K; K399R |
| | *4 | I264M |
| 2C9 | *1 | Wild-type |
| | *2 | R144C |
| | *3 | I359L |
| | *4 | 45325 T > C |
| | *5 | D360E |
| 2C18 | ml | T204A |
| | m2 | A460T |
| 2C19 | 1A | Wild-type |
| | *1B | I331V |
| | *2A | Splicing defect |
| | *2B | Splicing defect; E92D |
| | *3 | New stop codon 636G > A |
| | *4 | GTG initiation codon, 1 A > G |
| | *5(A, B) | 1297 C > T, amino acid change (R433W) |
| | *6 | 395G > A, amino acid change (R132Q) |
| | *7 | IVS5 + 2T > A, splicing defect |
| | *8 | 358T > C, amino acid change (W120R) |
| 2D6 | *1A | Wild-type |
| | *2 | G1661C, C2850T |
| | *2N | Gene duplication |
| | *3 | A2549 deletion |
| | *4 | G1846A |
| | *5 | Gene deletion |
| | *6 | T1707 deletion |
| | *7 | A2935C |
| | *8 | G1758T |
| | *10 | C100T |
| | *12 | G124A |
| | *17 | C1023T, C2850T |
| | *35 | G31A |
| 2E1 | *1A | Wild-type |
| | *1C, *1D | (6 or 8 bp repeats) |
| | *2 | G1132A |
| | *4 | G476A |
| | *5 | G(−1293)C |
| | *5 | C(−1053)T |
| | *7 | T(−333)A |
| | *7 | G(−71)T |
| | *7 | A(−353)G |
| 3A4 | *1A | Wild-type |
| | *1B | A(−392)G |
| | *2 | Amino acid change (S222P) |
| | *5 | Amino acid change (P218R) |
| | *6 | Frameshift, 831 ins A |
| | *12 | Amino acid change (L373F) |
| | *13 | Amino acid change (P416L) |
| | *15A | Amino acid change (R162Q) |
| | *17 | Amino acid change (F189S, decreased) |
| | *18A | Amino acid change (L293P, increased) |
| 3A5 | *1A | Wild-type |
| | *3 | A6986G |
| | *5 | T12952C |
| | *6 | G14960A |

### V. Algorithm

The step of selecting the medication or the optimal dose of the medication can further comprise using an algorithm. Based on the algorithm, medication profiles may be provided for a given subject based on the subject's genotype, allowing a clinician to determine the medication or an optimal dose of the medication without the trial and error of determining if the subject will respond or tolerate a particular drug. The methods involve use of the primers and probes of the present invention to determine the individual's genotype at least for CYP2C9 and VKORC1, and optionally, other genes including, but not limited to genes involved in drug metabolism. Other factors such as age and height of the subject are taken into consideration using the algorithm.

In one embodiment, the present invention provides a method for selecting an optimal dose of a medication for a human subject. The method comprises:

a) genotyping CYP2C9, VKORC1, or both to determine a genotype and, optionally, genotyping at least one additional gene; and

b) selecting the optimal dose of the medication based on the genotyping of step a), wherein the selecting further comprises using an algorithm based on the subject's CYP2C9 and/or VKORC1 genetic polymorphism, and one or more characteristics of the subject.

In one embodiment, the drug to be dosed is warfarin, preferably S-warfarin. By genotyping at least CYP2C9 and VKORC1 polymorphisms, one may properly dose warfarin.

In another embodiment, the subject to be dosed is screened for one or more SNPs in both the CYP2C9 gene and the VKORC1 gene.

In one embodiment, the method uses one or more of the CYP2C9 specific primers described herein, and any primer specific for an SNP in VKORC1.

In other embodiments, the method uses one or more of the VKORC1 specific primers described herein, and any primer specific for an SNP in CYP2C9.

In one embodiment, the present invention provides a method for determining an optimal dose of warfarin for a human subject. The method comprises:

a) genotyping CYP2C9, VKORC1, or both to determine a genotype and, optionally, genotyping at least one additional gene; and

b) selecting the optimal dose of warfarin based on the genotyping of step a), wherein the selecting further comprises using an algorithm based on the subject's CYP2C9 and/or VKORC1 genetic polymorphism, and one or more characteristics of the subject.

The method includes using the primers and probes of the present invention to determine the individual's genotype for CYP2C9 and VKORC1. Other characteristics of the subject such as, for example, age and height are taken into consideration using this dosing algorithm.

An algorithm based on the impact of CYP2C9 and VKORC1 genetic polymorphism and subject characteristics upon warfarin dose requirements is described in Sconce et al., (2005) Blood, 106:2329-33, the content of which is incorporated herein by reference in its entirety.

In one embodiment, the algorithm comprises √Dose (i.e., square root of dose)=0.628−0.0135(age)−0.240(CYP*2)−0.370(CYP*3)−0.241(VKORC1 1173)+0.24(VKORC1 3730)+0.0162(height). For the algorithm, age is determined in years; height in centimeters; and the input values for CYP*2 and CYP*3 genotype is 0, 1 or 2 according to the number of CYP*2 or CYP*3 alleles present; the VKORC1 1173: input is 1 for 1173CC, 2 for 1173CT, and 3 for 1173TT; VKORC1 3730 input is 0 for 3730GG, 0 for 3730GA, and 1 for 3730AA.

### VI. Articles of Manufacture

In other aspects, the present invention provides an article of manufacture (e.g., a kit). The article of manufacture can be developed using the nucleic acid sequences disclosed herein. These sequences can be used as primers in nucleic acid amplification reactions, and/or as probes in a nucleic acid hybridization method. The article of manufacture is useful for determining a subject's genotype. Components in the article of manufacture can either be obtained commercially or made according to well known methods in the art. In addition, the components of the article of manufacture can be in solution or lyophilized as appropriate.

In one embodiment, the components are in the same compartment, and in another embodiment, the components are in separate compartments. In the preferred embodiment, the article of manufacture further comprises instructions for use.

Optionally, the article of manufacture also may comprise buffers and other reagents necessary for PCR (e.g., DNA polymerase or nucleotides). The article of manufacture also may contain one or more primer pairs (e.g., 5, 10, 15, or 20 primer pairs), such that multiple nucleic acid products can be generated.

In other embodiments, an articles of manufacture comprises populations of the polynucleotides of the present invention immobilized on a substrate. Suitable substrates provide a base for the immobilization of the present polynucleotides, and in some embodiments, allow immobilization of the polynucleotides into discrete regions. In embodiments in which the substrate includes a plurality of discrete regions, different populations of isolated polynucleotides may be immobilized in each discrete region. The different populations of polynucleotides independently may include polynucleotides for detecting one or more of the CYP2C9 or VKORC1 alleles described herein.

Suitable substrates may be of any shape or form and can be constructed from, for example, glass, silicon, metal, plastic, cellulose or a composite. For example, a suitable substrate may include a multi-well plate or membrane, a glass slide, a chip, or polystyrene or magnetic beads. Polypeptides may be synthesized in situ, immobilized directly on the substrate, or immobilized via a linker, including by covalent, ionic, or physical linkage. Linkers for immobilizing nucleic acids and polypeptides, including reversible or cleavable linkers, are known in the art. See, e.g., U.S. Pat. No. 5,451,683 and WO 98/20019.

The invention has been described with reference to various specific and preferred embodiments and techniques. However, it should be understood that many variations and modifications may be made while remaining within the spirit and scope of the invention.

The following examples are provided for illustration only.

### EXAMPLES

### Example 1

### Real-Time PCR Method for Detection of Mutations within CYP2C9 and VKORC1

A real-time multiplex PCR assay was performed to identify two clinically relevant mutations in CYP2C9 (*2 and *3) and two mutations in VKORC1 (1173C>T and 3730G>A). Bi-directional DNA sequencing served as the method of comparison for the real-time multiplex assay.

Matched blood and saliva samples were obtained from 100 properly consented human subjects of known ethnicity. Each subject had whole blood drawn into a lavender-top EDTA BD Vacutainer® blood collection tube (BD Diagnostics, Sparks, Md.) and also provided a saliva sample using the Oragene™ DNA Self Collection Kit (DNA Genotek Inc., Ontario, Canada). Blood samples were processed using the PUREGENE® DNA Purification Kit (Gentra Systems, Minneapolis, Minn.).

Genomic DNA from saliva was prepared according to the manufacturer's (i.e., DNA Genotek Inc., Ontario, Canada) protocol, which included sample incubation at 50° C. for 1 hour, protein precipitation at −20° C. for 10 minutes, DNA precipitation for 10 minutes, and DNA pellet rehydration. Purified DNA from blood and saliva was quantitated by relative fluorescence intensity (RFI) assay.

The multiplex assays were run on the Cepheid SmartCycler® II using the prepared genomic DNA at a concentration of about 5 ng/μl to about 500 ng/μl. The assay consisted of two reaction tubes, with each tube containing mutant and wild-type primer and probe sets for two gene alleles.

As shown in Tables 2 and 3, the fluorogenic probes were labeled with Fam™, Cal Orange 560™, Cal Red 610™, or Quasar 670™ (Biosearch Technologies, Novato, Calif.) for minimal cross talk between emission wavelengths. Further, “black hole” quenchers, BHQ-1™ or BHQ-2™ (Biosearch Technologies, Novato, Calif.), were used as quenchers to prevent fluorescence until a hybridization event occurs.

TABLE 2
Polynucleotides (i.e., Primers and Probes) used in CYP2C9 genotyping.

| Name | Sequence | Label¹ | picomole |
|---|---|---|---|
| A | B | C | D |
| CYP2C9-34511-F | 5′-ccc ctg aat tgc tac aac aaa tgt | Fl: None | 50 |
| Forward primer | g-3′ | Q: None | |
| | (SEQ ID NO: 1) | | |
| CYP2C9-34511-R | 5′-gac ttc gaa aac atg gag tgc a-3′ | Fl: None | 50 |
| Reverse primer | (SEQ ID NO: 2) | Q: None | |
| CYP2C9-34511-R₂ | 5′-gac ttc gaa aac atg gag ttg ca-3′ | Fl: None | 50 |
| Reverse primer | (SEQ ID NO: 17) | Q: None | |
| CYP2C9*3 MUT | 5′-cag aga tac ctt gac ctt c-3′ | Fl: Cal Red 610 | 40 |
| Probe | (SEQ ID NO: 3) | Q: BHQ-2 | |
| CYP2C9*3 WT | 5′-cca gag ata cat tga cct tc-3′ | Fl: Quasar 670 | 10 |
| Probe | (SEQ ID NO: 4) | Q: BHQ-2 | |
| CYP2C9*2 F | 5′-ctc atg acg ctg cgg aat tt-3′ | Fl: None | 30 |
| Forward primer | (SEQ ID NO: 5) | Q: None | |
| CYP2C9*2 R | 5′-gaa gat agt agt cca gta agg tca | Fl: None | 30 |
| Reverse primer | gtg ata tg-3′ | Q: None | |
| | (SEQ ID NO: 6) | | |
| CYP2C9 *2 WT | 5′-cat tga gga ccg tgt tca-3′ | Fl: FAM | 10 |
| Probe | (SEQ ID NO: 7) | Q: BHQ-1 | |
| CYP2C9*2 MUT | 5′-cat tga gga ctg tgt tca a-3′ | Fl: Cal Orange | 20 |
| Probe | (SEQ ID NO: 8) | 560 | |
| | | Q: BHQ-1 | |

¹Label: F1 = 5′fluorophore; Q = 3′quencher.

TABLE 3
Polynucleotides (i.e., Primers and Probes) used in VKORC1 genotyping.

| Name | Sequence | Label¹ | picomole |
|---|---|---|---|
| A | B | C | D |
| VKORC1 1173 F | 5′-atc ctg acg tgg cca aag g-3′ | Fl: None | 12.5 |
| Forward primer | (SEQ ID NO: 9) | Q: None | |
| VKORC1 1173 R | 5′-cca cct ggg cta tcc tct gtt-3′ | Fl: None | 12.5 |
| Reverse primer | (SEQ ID NO: 10) | Q: None | |
| VKORCI 1173WT | 5′-cca gga gat cat cga ccc ttg | Fl: FAM | 6.25 |
| Probe | gac-3′ | Q: BHQ-1 | |
| | (SEQ ID NO: 11) | | |
| VKORCI 1173MUT | 5′-cca gga gat cat cga ctc ttg | Fl: Cal Orange 560 | 6.25 |
| Probe | gac tag g-3′ | Q: BHQ-1 | |
| | (SEQ ID NO: 12) | | |
| VKORC1 3730F | 5′-ccc tag atg tgg ggc ttc tag | Fl: None | 12.5 |
| Forward primer | att a-3′ | Q: None | |
| | (SEQ ID NO: 13) | | |
| VKORCI 3730R | 5′-agc gtg tgg cac att tgg t-3′ | Fl: None | 12.5 |
| Reverse primer | (SEQ ID NO: 14) | Q: None | |
| VKORC1 3730MUT | 5′-ctc ctg cca tac cca cac atg | Fl: Cal Red 610 | 7.0 |
| Probe | aca at-3′ | Q: BHQ-2 | |
| | (SEQ ID NO: 15) | | |
| VKORC1 3730WT | 5′-ctc ctg cca tac ccg cac atg | Fl: Quasar 670 | 9.0 |
| Probe | a-3′ | Q: BHQ-2 | |
| | (SEQ ID NO: 16) | | |

¹Label: F1 = 5′fluorophore; Q = 3′quencher.

The PCR reaction was performed using the SmartMix™ HM PCR Master Mix (Cepheid, Sunnyvale, Calif.) and the polynucleotides shown in Tables 2 and 3 using the picomole amounts shown in column D. For CYP2C9 genotyping, samples were subjected to 1 cycle of 15 sec at 95° C. followed by 35 cycles of 1 sec at 95° C., 6 sec at 58° C., and 6 sec at 72° C. For VKORC1 genotyping, samples were subjected to 1 cycle of 15 sec at 95° C. followed by 35 cycles of 1 sec at 95° C., 6 sec at 62° C., and 6 sec at 72° C. Due to the rapid thermocycling parameters, the entire assay protocol was completed in less than twenty minutes.

Bi-directional sequencing was performed following standard sequencing techniques. Cycle sequencing reactions were performed by using ABI BigDye® Terminator Version 1.1 and the ABI PRISM® 3130xl Genetic Analyzer (Applied Biosystems, Foster City, Calif.). Genomic DNA was isolated from whole blood using PUREGENE® DNA Purification Kit (Gentra Systems, Minneapolis, Minn.). PCR amplicons containing region of interest (SNP) were generated using specific sense and anti-sense primers and standard PCR techniques, and purified using the Qiagen Miniprep Kit (Qiagen, Valencia, Calif.). Cycle sequencing reactions were performed using amplicon with region of interest and the ABI BigDye® Terminator Version 1.1 Kit reagents (Applied Biosystems, Foster City, Calif.).

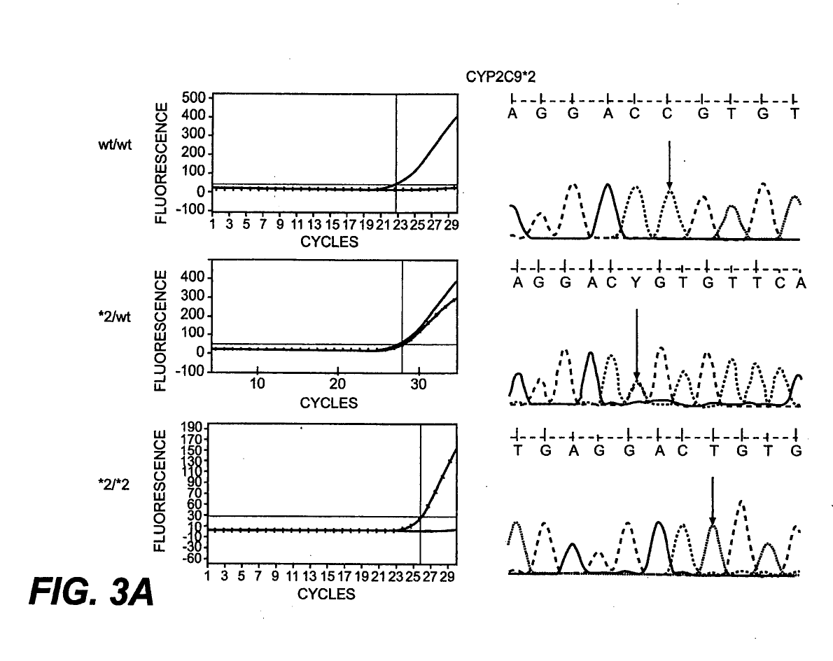

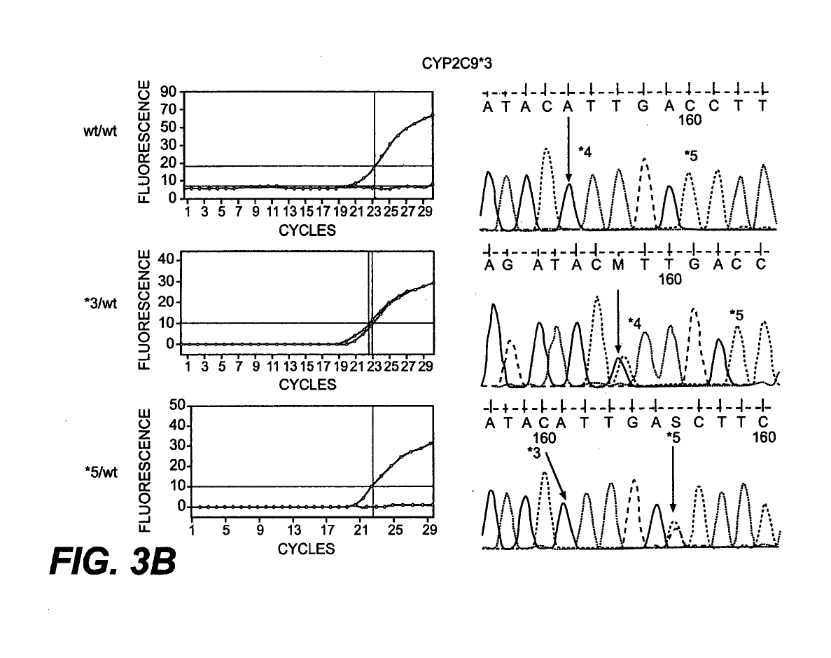

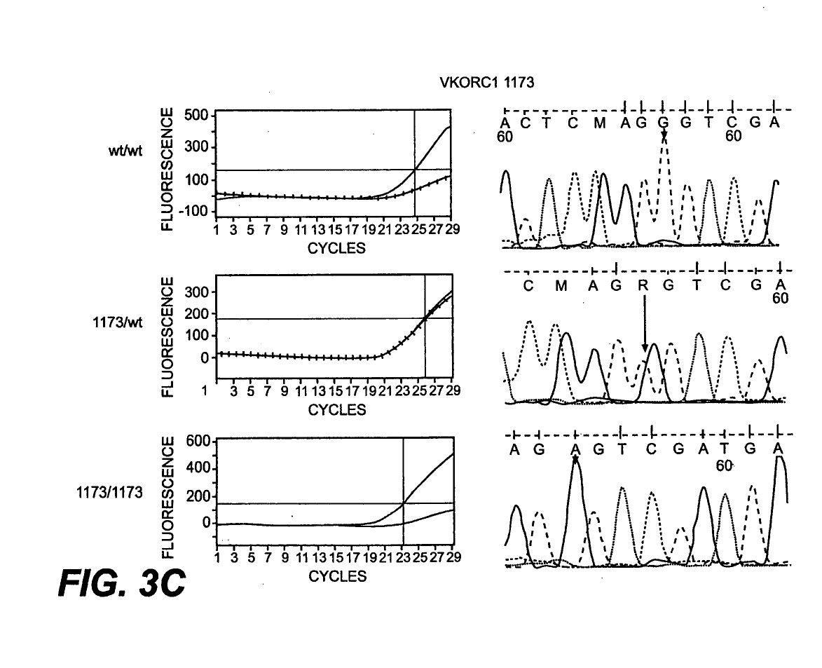

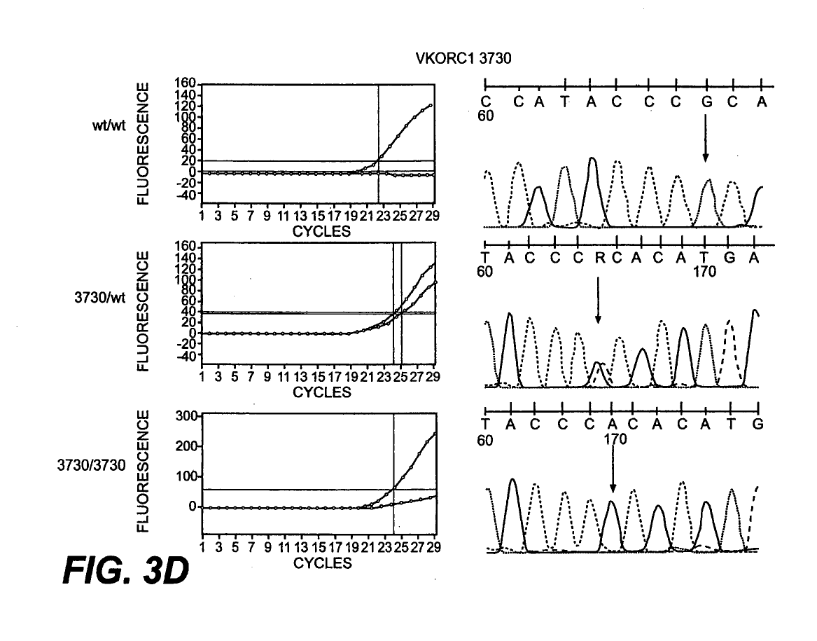

There was complete concordance between the bi-directional sequencing and the real-time multiplex assay as shown in FIG. 3(a-d). There were zero miscalls out of a total of 94 assay calls. There were six samples that gave a “no call” result. A “call” is described as a real-time PCR reaction whose amplification plots do not hit the threshold florescence values by cycle 33 of the run. Upon further investigation, it was discovered that the “no call” samples were of a very low DNA concentration (<1 ng/μl).

### Example 2

### Comparison Between the Use of Blood and Saliva for Genotype Determination

Genotype results obtained using genomic DNA from whole blood and saliva from the same subject were compared. For each real-time multiplex PCR assay run, a cycle threshold (Ct) value was obtained where Ct is defined as the cycle number at which fluorescence passes the fixed threshold value. A delta Ct comparison method was used to determine genotype, where delta Ct=Wild-type Ct value−Mutant Ct value.

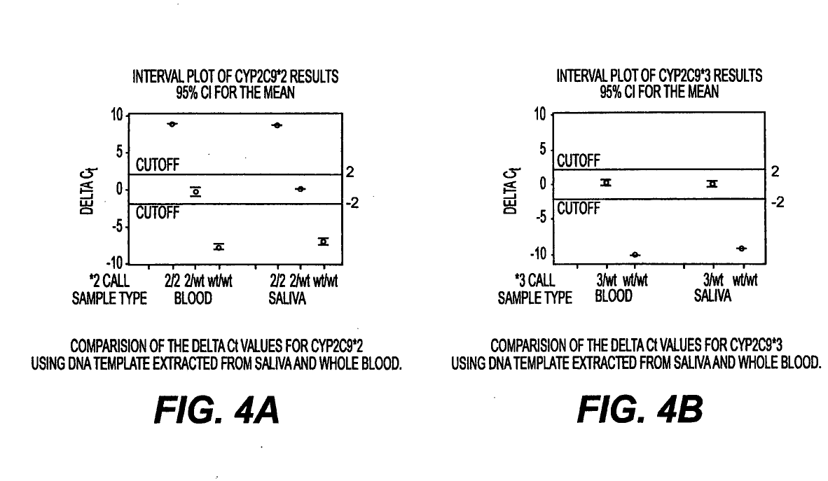

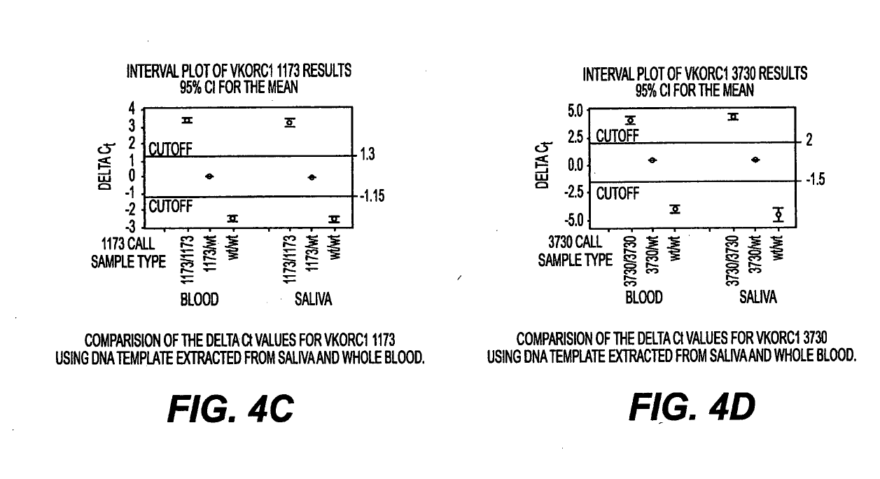

The data obtained from the comparison study was inserted into Minitab™ 14 statistical software package (Minitab Inc., State College, Pa.) and analyzed for trends associated with the delta Ct value. As shown in FIG. 4(a-d), there was no statistically significant difference (95% confidence intervals) between the delta Ct values for DNA derived from blood and the DNA derived from saliva for both the CYP2C9 & VKORC1 assays. Accordingly, there was no statistically significant difference between the genotype calls as determined by the assay.

Table 4 is a tabular comparison of the genotypes frequencies from the NCBI SNP database (ww.ncbi.nlm.nih.gov/SNP/) and the genotypes obtained from the blood versus saliva study. The Rapid Genotyping Assay for CYP2C9 & VKORC1 described herein was performed on the matched set of one hundred blood DNA samples and one hundred saliva DNA samples. As shown in Table 4, there were no major differences between the documented frequencies and the frequencies derived from our assays.

TABLE 4
Genotype Frequency.

| | | Caucasian | Blood | Saliva |
|---|---|---|---|---|
| Allele | Genotype | Frequency¹ | Frequency | Frequency |
| VKORC1: 1173 | wt/wt | 37% | 44% | 44% |
| | 1173/wt | 47% | 43% | 43% |
| | 1173/1173 | 16% | 13% | 13% |
| VKORC1: 3730 | wt/wt | 46% | 43% | 43% |
| | 3730/wt | 39% | 43% | 43% |
| | 3730/3730 | 15% | 14% | 14% |
| CYP2C9: *2 | wt/wt | 85% | 82% | 82% |
| | *2/wt | 13% | 17% | 17% |
| | *2/*2 | 2% | 1% | 1% |
| CYP2C9: *3 | wt/wt | 92% | 87% | 87% |
| | *3/wt | 8% | 13% | 13% |
| | *3/*3 | <0.1% | 0% | 0% |

¹Caucasian frequencies are from the NCBI SNP database (www.ncbi.nlm.nih.gov/SNP/).

### Example 3

### Reverse Primer Comparison for Detection of the *3 Mutation within CYP2C9

A subsequent study was performed in order to demonstrate the equivalency of two primer sequences used for generation of the CYP2C9*3 amplicon product for the CYP2C9*3 reaction. The original primer sequence was used throughout the clinical investigation in which three different laboratories tested the primer & probe formulations and will therefore be referred to as the “clinical” primer. The primer possessing the full length sequence (“rework” primer), as determined by BLAST alignment, was tested side-by-side with the “clinical” primer.

The multiplex assays were run on the Cepheid SmartCycler® II using the prepared genomic DNA at a concentration of 10 ng/μl. The assay consisted of two reaction tubes, with each tube containing mutant and wild-type primer and probe sets for two gene alleles. Thirty (30) unique DNA samples (previously isolated from blood samples) were tested using both the “clinical” primer/probe mix and the “rework” primer/probe mix. In addition, testing was performed on two different days for each of the samples, to yield a total of 60 results for each primer/probe mix. The samples had the following CYP2C9*3 genotypes: fifteen (15)*3/wt samples; three (3)*3/*3 samples, and twelve (12) wt/wt samples.

As shown in Tables 2 and 3, the fluorogenic probes were labeled with Fam™, Cal Orange 560™, Cal Red 610™, or Quasar 670™ (Biosearch Technologies, Novato, Calif.) for minimal cross talk between emission wavelengths. Further, “black hole” quenchers, BHQ-1™ or BHQ-2™ (Biosearch Technologies, Novato, Calif.), were used as quenchers to prevent fluorescence until a hybridization event occurs.

TABLE 5
Polynucleotides (i.e., Primers and Probes) used in CYP2C9 genotyping.

| Name | Sequence | Label¹ | picomole |
|---|---|---|---|
| A | B | C | D |
| CYP2C9-34511-F | 5′-ccc ctg aat tgc tac aac aaa tgt g-3′ | Fl: None | 50 |
| Forward primer | (SEQ ID NO: 1) | Q: None | |
| CYP2C9-34511-R | 5′-gac ttc gaa aac atg gag tgc a-3′ | Fl: None | 50 |
| Reverse primer | (SEQ ID NO: 2) | Q: None | |
| CYP2C9-34511-R2 | 5′-gac ttc gaa aac atg gag ttg ca-3′ | Fl: None | 50 |
| Reverse primer | (SEQ ID NO: 17) | Q: None | |
| CYP2C9*3 MUT | 5′-cag aga tac ctt gac ctt c-3′ | Fl: Cal Red | 40 |
| Probe | (SEQ ID NO: 3) | 610 | |
| | | Q: BHQ-2 | |
| CYP2C9*3 WT | 5′-cca gag ata cat tga cct tc-3′ | Fl: Quasar 670 | 10 |
| Probe | (SEQ ID NO: 4) | Q: BHQ-2 | |
| CYP2C9*2 F | 5′-ctc atg acg ctg cgg aat tt-3′ | Fl: None | 30 |
| Forward primer | (SEQ ID NO: 5) | Q: None | |
| CYP2C9*2 R | 5′-gaa gat agt agt cca gta agg tca gtg | Fl: None | 30 |
| Reverse primer | ata tg-3′ | Q: None | |
| | (SEQ ID NO: 6) | | |
| CYP2C9 *2 WT | 5′-cat tga gga ccg tgt tca-3′ | Fl: FAM | 10 |
| Probe | (SEQ ID NO: 7) | Q: BHQ-1 | |
| CYP2C9*2 MUT | 5′-cat tga gga ctg tgt tca a-3′ | Fl: Cal Orange | 20 |
| Probe | (SEQ ID NO: 8) | 560 | |
| | | Q: BHQ-1 | |

¹Label: F1 = 5′fluorophore; Q = 3′quencher.

TABLE 6
Polynucleotides (i.e., Primers and Probes) used in VKORC1 genotyping.

| Name | Sequence | Label¹ | picomole |
|---|---|---|---|
| A | B | C | D |
| VKORC1 1173 F | 5′-atc ctg acg tgg cca aag g-3′ | Fl: None | 12.5 |
| Forward primer | (SEQ ID NO: 9) | Q: None | |
| VKORC1 1173 R | 5′-cca cct ggg cta tcc tct gtt-3′ | Fl: None | 12.5 |
| Reverse primer | (SEQ ID NO: 10) | Q: None | |
| VKORC1 1173WT | 5′-cca gga gat cat cga ccc ttg | Fl: FAM | 6.25 |
| Probe | gac-3′ | Q: BHQ-1 | |
| | (SEQ ID NO: 11) | | |
| VKORC1 1173MUT | 5′-cca gga gat cat cga ctc ttg | Fl: Cal Orange | 6.25 |
| Probe | gac tag g-3′ | 560 | |
| | (SEQ ID NO: 12) | Q: BHQ-1 | |
| VKORC1 3730F | 5′-ccc tag atg tgg ggc ttc tag | Fl: None | 12.5 |
| Forward primer | att a-3′ | Q: None | |
| | (SEQ ID NO: 13) | | |
| VKORC1 3730R | 5′-agc gtg tgg cac att tgg t-3′ | Fl: None | 12.5 |
| Reverse primer | (SEQ ID NO: 14) | Q: None | |
| VKORC1 3730MUT | 5′-ctc ctg cca tac cca cac atg | Fl: Cal Red 610 | 7.0 |
| Probe | aca at-3′ | Q: BHQ-2 | |
| | (SEQ ID NO: 15) | | |
| VKORC1 3730WT | 5′-ctc ctg cca tac ccg cac atg | Fl: Quasar 670 | 9.0 |
| Probe | a-3′ | Q: BHQ-2 | |
| | (SEQ ID NO: 16) | | |

¹Label: F1 = 5′fluorophore; Q = 3′quencher.

The PCR reaction was performed using the SmartMix™ HM PCR Master Mix (Cepheid, Sunnyvale, Calif.) and the polynucleotides shown in Tables 2 and 3 using the picomole amounts shown in column D. For CYP2C9 genotyping, samples were subjected to 1 cycle of 15 sec at 95° C. followed by 35 cycles of 1 sec at 95° C., 6 sec at 58° C., and 6 sec at 72° C. For VKORC1 genotyping, samples were subjected to 1 cycle of 15 sec at 95° C. followed by 35 cycles of 1 sec at 95° C., 6 sec at 62° C., and 6 sec at 72° C. Due to the rapid thermocycling parameters, the entire assay protocol was completed in less than twenty minutes.

The end-point fluorescence and ΔCt values (change in cycle threshold values between mutant and wild-type probes) were evaluated for both primers. In all cases, there was no statistical difference between the data observed using the “clinical” primer and the “rework” primer.

### Example 4

### Cross-Platform Compatibility: Amplification and Detection of CYP2C9 & VKORC1 Alleles

The polynucleotides of the present invention were tested for cross-platform compatibility for detecting each of the 4 CYP2C9 alleles and the 4 VKORC1 alleles using the ABI 7500 real-time quantitative PCR instrument (Applied Biosystems, Foster City, Calif.). The PCR reaction was performed using a recombinant Taq polymerase PCR master mix. The FAM, Cy3, Texas Red, and Cy5 dye channels were utilized by the ABI 7500 instrument to detect fluorescence of the BioSearch dyes described in Tables 2 and 3 above.

Gentrisure™ Human Genomic Reference Control (ParagonDx LLC., Morrisville, N.C.) for CYP2C9*2/*3 and VKORC1 1173 CT/VKORC1 3730 GA were tested using the primers and probes for CYP2C9 and VKORC1 described in Table 7 & Table 8 (the final primer and probe amounts are shown in Column E). The PCR parameters were as follows: 1 cycle of 2 minutes at 95° C. followed by 35 cycles of 10 sec at 95° C., 25 sec at 60° C., and 15 sec at 72° C.

TABLE 7
Polynucleotides (i.e., Primers and Probes) used in CYP2C9 genotyping.

| Name | Sequence | Label¹ | picomole |
|---|---|---|---|
| A | B | C | D |
| CYP2C9-3-F | 5′-tgc aag aca gga gcc aca tg-3′ | Fl: None | 10.0 |
| Forward primer | (SEQ ID NO: 18) | Q: None | |
| CYP2C9-3-R | 5′-tta cct tgg gaa tga gat agt ttc | Fl: None | 10.0 |
| Reverse primer | tg-3′ | Q: None | |
| | (SEQ ID NO: 19) | | |
| CYP2C9*3 MUT | 5′-tcc aga gat acc ttg acc ttc tc-3′ | Fl: Cal Red 610 | 5.0 |
| Probe | (SEQ ID NO: 20) | Q: BHQ-2 | |
| CYP2C9*3 WT | 5′-tcc aga gat aca ttg acc ttc tc-3′ | Fl: Qupsar 670 | 5.0 |
| Probe | (SEQ ID NO: 21) | Q: BHQ-2 | |
| CYP2C9*2 F | 5′-ctc atg acg ctg cgg aat tt-3′ | Fl: None | 12.5 |
| Forward primer | (SEQ ID NO: 5) | Q: None | |
| CYP2C9*2 R | 5′-gaa gat agt agt cca gta agg tca | Fl: None | 12.5 |
| Reverse primer | gtg ata tg-3′ | Q: None | |
| | (SEQ ID NO: 6) | | |
| CYP2C9 *2 WT | 5′-cat tga gga ccg tgt tca-3′ | Fl: FAM | 2.5 |
| Probe | (SEQ ID NO: 7) | Q: BHQ-1 | |
| CYP2C9*2 MUT | 5′-cat tga gga ctg tgt tca a-3′ | Fl: Cal Orange 560 | 10.0 |
| Probe | (SEQ ID NO: 8) | Q: BHQ-1 | |

¹Label: F1 = 5′fluorophore; Q = 3′quencher.

TABLE 8
Polynucleotides (i.e., Primers and Probes) used in VKORC1 genotyping.

| Name | Sequence | Label¹ | picomole |
|---|---|---|---|
| A | B | C | D |
| VKORC1 1173 F | 5′-atc ctg acg tgg cca aag g-3′ | Fl: None | 7.5 |
| Forward primer | (SEQ ID NO: 9) | Q None | |
| VKORC1 1173 R | 5′-cca cct ggg cta tcc tct gtt-3′ | Fl: None | 7.5 |
| Reverse primer | (SEQ ID NO: 10) | Q: None | |
| VKORC1 1173WT | 5′-cca gga gat cat cga ccc ttg | Fl: FAM | 1.25 |
| Probe | gac-3′ | Q: BHQ-1 | |
| | (SEQ ID NO: 11) | | |
| VKORC1 1173MUT | 5′-cca gga gat cat cga ctc ttg | Fl: Cal Orange 560 | 6.25 |
| Probe | gac tag g-3′ | Q: BHQ-1 | |
| | (SEQ ID NO: 12) | | |
| VKORC1 3730F | 5′-ccc tag atg tgg ggc ttc tag | Fl: None | 5.0 |
| Forward primer | att a-3′ | Q: None | |
| | (SEQ ID NO: 13) | | |
| VKORC1 3730R | 5′-agc gtg tgg cac att tgg t-3′ | Fl: None | 5.0 |
| Reverse primer | (SEQ ID NO: 14) | Q: None | |
| VKORC1 3730MUT | 5′-cte ctg cca tac cca cac atg | Fl: Cal Red 610 | 1.25 |
| Probe | aca at-3′ | Q: BHQ-2 | |
| | (SEQ ID NO: 15) | | |
| VKORC1 3730WT | 5′-ctc ctg cca tac ccg cac atg | Fl: Quasar 670 | 3.75 |
| Probe | a-3′ | Q: BHQ-2 | |
| | (SEQ ID NO: 16) | | |

¹Label: F1 = 5′fluorophore; Q = 3′ quencher.

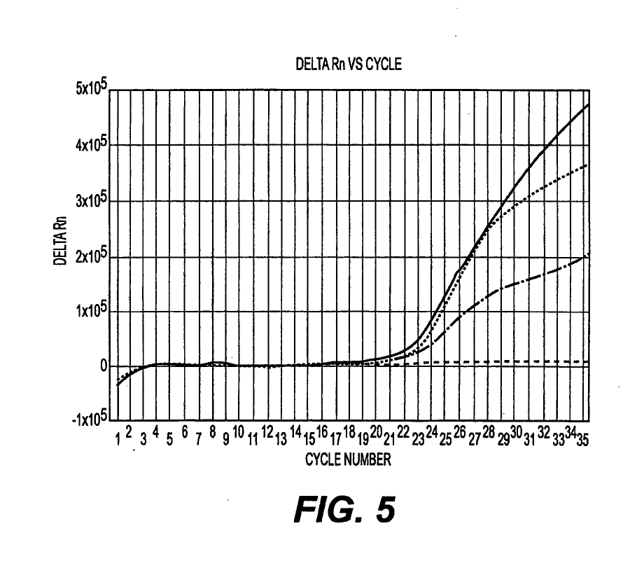

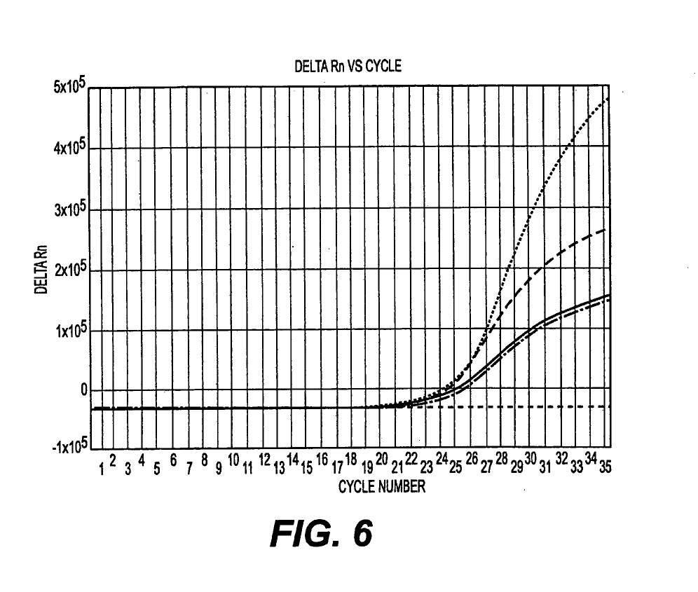

The real-time PCR amplification plots that were obtained are shown in FIG. 5 and FIG. 6. FIG. 5 shows two separate runs in which the CYP2C9*2/*3 sample was tested using the CYP2C9*2/*3 master mix containing CYP2C9*2 primers, CYP2C9*3 primers and both mutant and wild-type probes for CYP2C9*2 and *3. Since both the wild-type and mutant probes demonstrate fluorescence above the threshold value (horizontal line), the CYP2C9 genotype was correctly called as CYP2C9*2/*3. FIG. 6 shows two separate runs in which the VKORC1 1173 CT/3730 GA sample was tested using the VKORC 1 1173/3730 master mix containing VKORC1 1173 primers, VKORC1 3730 primers and both mutant and wild-type probes for VKORC1 1173 and 3730. Since both the wild-type and mutant probes demonstrate fluorescence above the threshold value (horizontal line), the VKORC1 genotype was correctly called as VKORC1 1173 CT/3730 GA.

### Example 5

### Cross-Platform Compatibility: Amplification and Detection of CYP2C9 & VKORC1 Alleles

The polynucleotides of the present invention were tested for cross-platform compatibility for detecting each of the 4 CYP2C9 alleles and the 4 VKORC1 alleles using the Stratagene Mx3005P real-time quantitative PCR instrument (Stratagene, La Jolla, Calif.). The PCR reaction was performed using the recombinant Taq polymerase PCR master mix. The FAM, Cy3, Texas Red, and Cy5 dye channels were utilized by the Stratagene Mx3005P instrument to detect fluorescence of the BioSearch dyes described in Tables 7 and 8 above.

Gentrisure™ Human Genomic Reference Control (ParagonDx LLC., Morrisville, N.C.) for CYP2C9*2/*3 and VKORC1 1173 CT/VKORC1 3730 GA were tested using the primers and probes for CYP2C9 and VKORC1 described in Table 9 & Table 10 (the final primer and probe amounts are shown in Column E).The PCR parameters were as follows: 1 cycle of 2 minutes at 95° C. followed by 35 cycles of 10 sec at 95° C., 25 sec at 60° C., and 15 sec at 72° C.

TABLE 9
Polynucleotides (i.e., Primers and Probes) used in CYP2C9 genotyping.

| Name | Sequence | Label¹ | picomole |
|---|---|---|---|
| A | B | C | D |
| CYP2C9-3-F | 5′-tgc aag aca gga gcc aca tg-3′ | Fl: None | 18.75 |
| Forward primer | (SEQ ID NO: 18) | Q: None | |
| CYP2C9-3-R | 5′-tta cct tgg gaa tga gat agt ttc | Fl: None | 18.75 |
| Reverse primer | tg-3′ | Q: None | |
| | (SEQ ID NO: 19) | | |
| CYP2C9*3 MUT | 5′-tcc aga gat acc ttg acc ttc tc-3′ | Fl: Cal Red 610 | 5.0 |
| Probe | (SEQ ID NO: 20) | Q: BHQ-2 | |
| CYP2C9*3 WT | 5′-tcc aga gat aca ttg acc ttc tc-3′ | Fl: Quasar 670 | 5.0 |
| Probe | (SEQ ID NO: 21) | Q: BHQ-2 | |
| CYP2C9*2 F | 5′-ctc atg acg ctg cgg aat tt-3′ | Fl: None | 18.75 |
| Forward primer | (SEQ ID NO: 5) | Q: None | |
| CYP2C9*2 R | 5′-gaa gat agt agt cca gta agg tca | Fl: None | 18.75 |
| Reverse primer | gtg ata tg-3′ | Q: None | |
| | (SEQ ID NO: 6) | | |
| CYP2C9 *2 WT | 5′-cat tga gga ccg tgt tca-3′ | Fl: FAM | 1.25 |
| Probe | (SEQ ID NO: 7) | Q: BHQ-1 | |
| CYP2C9*2 MUT | 5′-cat tga gga ctg tgt tca a-3′ | Fl: Cal Orange 560 | 10.0 |
| Probe | (SEQ ID NO: 8) | Q: BHQ-1 | |

¹Label: F1 = 5′fluorophore; Q = 3′quencher.

TABLE 10
Polynucleotides (i. e. , Primers and Probes) used in VKORC1 genotyping.

| Name | Sequence | Label¹ | picomole |
|---|---|---|---|
| A | B | C | D |
| VKORC1 1173 F | 5′-atc ctg acg tgg cca aag g-3′ | Fl: None | 18.75 |
| Forward primer | (SEQ ID NO: 9) | Q: None | |
| VKORC1 1173 R | 5′-cca cct ggg cta tcc tct gtt-3′ | Fl: None | 18.75 |
| Reverse primer | (SEQ ID NO: 10) | Q: None | |
| VKORC1 1173 WT | 5′-cca gga gat cat cga ccc ttg | Fl: FAM | 0.63 |
| Probe | gac-3′ | Q: BHQ-1 | |
| | (SEQ ID NO: 11) | | |
| VKORC1 1173MUT | 5′-cca gga gat cat cga ctc ttg | Fl: Cal Orange 560 | 7.5 |
| Probe | gac tag g-3′ | Q: BHQ-1 | |
| | (SEQ ID NO: 12) | | |
| VKORC1 3730F | 5′-ccc tag atg tgg ggc ttc tag | Fl: None | 18.75 |
| Forward primer | att a-3′ | Q: None | |
| | (SEQ ID NO: 13) | | |
| VKORC1 3730R | 5′-agc gtg tgg cac att tgg t-3′ | Fl: None | 18.75 |
| Reverse primer | (SEQ ID NO: 14) | Q: None | |
| VKORC1 3730MUT | 5′-ctc ctg cca tac cca cac atg | Fl: Cal Red 610 | 1.25 |
| Probe | aca at-3′ | Q: BHQ-2 | |
| | (SEQ ID NO: 15) | | |
| VKORC1 3730WT | 5′-ctc ctg cca tac ccg cac atg | Fl: Quasar 670 | 5.0 |
| Probe | a-3′ | Q: BHQ-2 | |
| | (SEQ ID NO: 16) | | |

¹Label: F1 = 5′fluorophore; Q = 3′quencher.

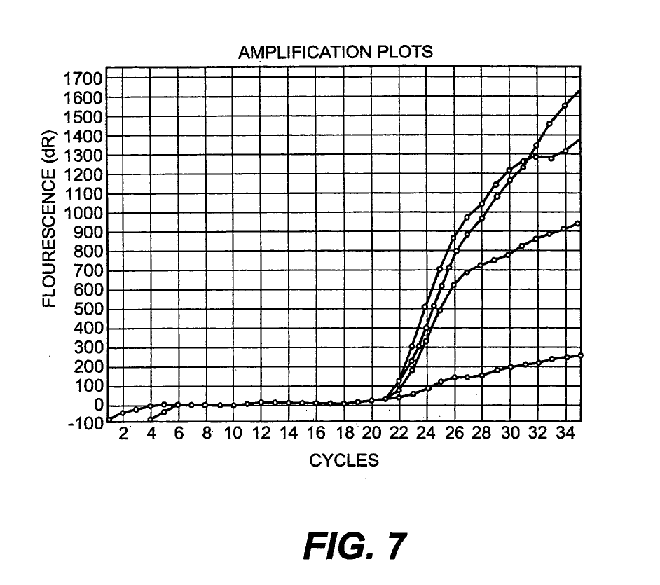

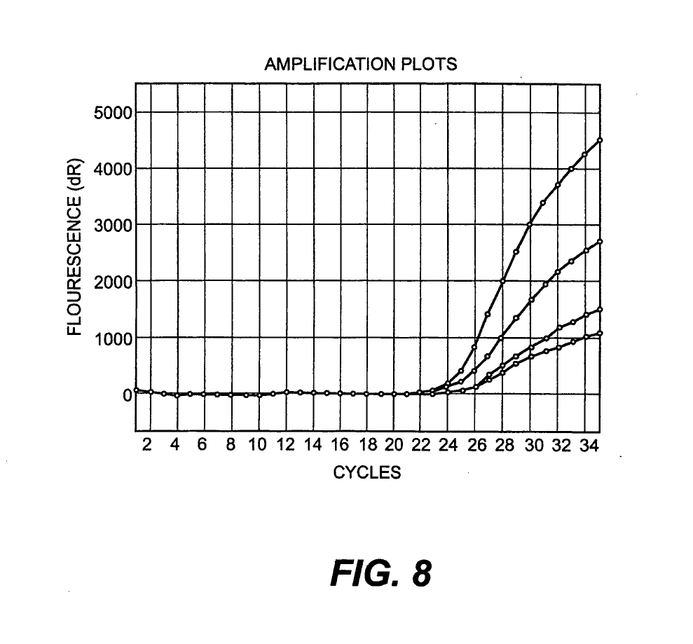

The real-time PCR amplification plots that were obtained are shown in FIG. 7 and FIG. 8. FIG. 7 shows two separate runs in which the CYP2C9*2/*3 sample was tested using the CYP2C9*2/*3 master mix containing CYP2C9*2 primers, CYP2C9*3 primers and both mutant and wild-type probes for CYP2C9*2 and *3. Since both the wild-type and mutant probes demonstrate fluorescence above the threshold value (horizontal line), the CYP2C9 genotype was correctly called as CYP2C9*2/*3. FIG. 8 shows two separate runs in which the VKORC1 1173 CT/3730 GA sample was tested using the VKORC1 1173/3730 master mix containing VKORC1 1173 primers, VKORC1 3730 primers and both mutant and wild-type probes for VKORC1 1173 and 3730. Since both the wild-type and mutant probes demonstrate fluorescence above the threshold value (horizontal line), the VKORC1 genotype was correctly called as VKORC1 1173 CT/3730 GA.

That which is claimed is:

1. An isolated polynucleotide comprising a nucleotide sequence or complement thereof, wherein the nucleotide sequence is selected from the group consisting of:
(SEQ ID NO: 1)
5′-CCCCTGAATTGCTACAACAAATGTG-3′;
(SEQ ID NO: 2)
5′-GACTTCGAAAACATGGAGTGCA-3′;
(SEQ ID NO: 3)
5′-CAGAGATACCTTGACCTTC-3′;
(SEQ ID NO: 4)
5′-CCAGAGATACATTGACCTTC-3′;
(SEQ ID NO: 5)
5′-CTCATGACGCTGCGGAATTT-3′;
(SEQ ID NO: 6)
5′-GAAGATAGTAGTCCAGTAAGGTCAGTGATATG-3′;
(SEQ ID NO: 7)
5′-CATTGAGGACCGTGTTCA-3′;
(SEQ ID NO: 8)
5′-CATTGAGGACTGTGTTCAA-3′;
(SEQ ID NO: 9)
5′-ATCCTGACGTGGCCAAAGG-3′;
(SEQ ID NO: 10)
5′-CCACCTGGGCTATCCTCTGTT′-3′;
(SEQ ID NO: 11)
5′-CCAGGAGATCATCGACCCTTGGAC-3′;
(SEQ ID NO: 12)
5′-CCAGGAGATCATCGACTCTTGGACTAGG-3′;
(SEQ ID NO: 13)
5′-CCCTAGATGTGGGGCTTCTAGATTA-3′;
(SEQ ID NO: 14)
5′-AGCGTGTGGCACATTTGGT-3′;
(SEQ ID NO: 15)
5′-CTCCTGCCATACCCACACATGACAAT-3′;
(SEQ ID NO: 16)
5′-CTCCTGCCATACCCGCACATGA-3′;
(SEQ ID NO: 17)
5′-GACTTCGAAAACATGGAGTTGCA-3′,
(SEQ ID NO: 18)
5′-TGCAAGACAGGAGCCACATG-3′,
(SEQ ID NO: 19)
5′-TTACCTTGGGAATGAGATAGTTTCTG-3′,
(SEQ ID NO: 20)
5′-TCCAGAGATACCTTGACCTTCTC -3′,
(SEQ ID NO: 21)
5′-TCCAGAGATACATTGACCTTCTC-3′,
wherein the isolated polynucleotide has a total nucleotide length of about 18 to about 50 nucleotides.

2. An isolated polynucleotide comprising a sequence consisting of at least 10, at least 18, at least 20, or at least 50 contiguous nucleotides or a complement thereof, wherein the contiguous nucleotides are contained in a nucleotide sequence selected from the group consisting of: (SEQ ID NO: 1), (SEQ ID NO: 2), (SEQ ID NO: 3), (SEQ ID NO: 4), (SEQ ID NO: 5), (SEQ ID NO: 6), (SEQ ID NO: 7), (SEQ ID NO: 8), (SEQ ID NO: 9), (SEQ ID NO: 10), (SEQ ID NO: 11), (SEQ ID NO: 12), (SEQ ID NO: 13), (SEQ ID NO: 14), (SEQ ID NO: 15), (SEQ ID NO: 16), (SEQ ID NO: 17), (SEQ ID NO: 18), (SEQ ID NO: 19), (SEQ ID NO: 20), and (SEQ ID NO: 21).

3. The isolated polynucleotide of claim 2, wherein the contiguous nucleotides are contained in a nucleotide sequence selected from the group consisting of (SEQ ID NO: 3), (SEQ ID NO: 4), (SEQ ID NO: 7), (SEQ ID NO: 8), (SEQ ID NO: 20), (SEQ ID NO: 21), (SEQ ID NO: 11), (SEQ ID NO: 12), (SEQ ID NO: 15), and (SEQ ID NO: 16).

4. A set of primers comprising at least one primer pair, wherein the at least one primer pair comprises a first primer and a second primer each having a sequence consisting of at least 10, at least 18, at least 20, or at least 50 contiguous nucleotides or a complement thereof, wherein the contiguous nucleotides are, respectively, contained in a nucleotide sequence selected from the group consisting of:
(SEQ ID NO: 1) and (SEQ ID NO: 2);
(SEQ ID NO: 1) and (SEQ ID NO: 17);
(SEQ ID NO: 1) and (SEQ ID NO: 19);
(SEQ ID NO: 5) and (SEQ ID NO: 6);
(SEQ ID NO: 9) and (SEQ ID NO: 10);
(SEQ ID NO: 13) and (SEQ ID NO: 14);
(SEQ ID NO: 18) and (SEQ ID NO: 17); and
(SEQ ID NO:18) and (SEQ ID NO: 19),
wherein, optionally, the set of primers further comprises at least one additional primer pair other than the at least one primer pair.

5. An allele-specific primer pair comprising:
a) a first primer comprising a first primer nucleotide sequence selected from the group consisting of (SEQ ID NO: 1), (SEQ ID NO: 2), (SEQ ID NO: 5), (SEQ ID NO: 6), (SEQ ID NO: 9), (SEQ ID NO: 10), (SEQ ID NO: 13), (SEQ ID NO: 14), (SEQ ID NO: 17), (SEQ ID NO: 18) and (SEQ ID NO: 19), wherein the first primer has a total nucleotide length of about 18 to about 50 nucleotides; and
b) a second primer comprising a second primer nucleotide sequence or complement thereof, wherein the second primer nucleotide sequence is selected from the group consisting of (SEQ ID NO: 3), (SEQ ID NO: 4), (SEQ ID NO: 7), (SEQ ID NO: 8), (SEQ ID NO: 11), (SEQ ID NO: 12), (SEQ ID NO: 15), (SEQ ID NO: 16), (SEQ ID NO: 20) and (SEQ ID NO: 21), wherein the second primer has a total nucleotide length of about 18 to about 50 nucleotides.

6. An isolated polynucleotide conjugated to a detectable label, wherein the polynucleotide comprises a nucleotide sequence or complement thereof, wherein the nucleotide sequence is selected from the group consisting of (SEQ ID NO: 1), (SEQ ID NO: 2), (SEQ ID NO: 3), (SEQ ID NO: 4), (SEQ ID NO: 5), (SEQ ID NO: 6), (SEQ ID NO: 7), (SEQ ID NO: 8), (SEQ ID NO: 9), (SEQ ID NO: 10), (SEQ ID NO: 11), (SEQ ID NO: 12), (SEQ ID NO: 13), (SEQ ID NO: 14), (SEQ ID NO: 15), (SEQ ID NO: 16), (SEQ ID NO: 17), (SEQ ID NO: 18), (SEQ ID NO: 19), (SEQ ID NO: 20) and (SEQ ID NO: 21), wherein the isolated polynucleotide has a total nucleotide length of about 18 to about 50 nucleotides.

7. The isolated polynucleotide of claim 6, wherein the nucleotide sequence is selected from the group consisting of (SEQ ID NO: 3), (SEQ ID NO: 4), (SEQ ID NO: 7), (SEQ ID NO: 8), (SEQ ID NO: 11), (SEQ ID NO: 12), (SEQ ID NO: 15), (SEQ ID NO: 16), (SEQ ID NO: 20) and (SEQ ID NO: 21).

8. The isolated polynucleotide of claim 6 wherein the polynucleotide is conjugated to a detectable label comprising a 5′-fluorophore/3′-quencher selected from Cal Red 610/BHQ-2, Quasar 670/BHQ-2, FAM/BHQ-1, Cal Orange 560/BHQ-1, or Cal Orange 560, BHQ-1.

9. An isolated polynucleotide conjugated to a detectable label, wherein the polynucleotide consists essentially of a nucleotide sequence or complement thereof, wherein the nucleotide sequence is selected from the group consisting of (SEQ ID NO: 1), (SEQ ID NO: 2), (SEQ ID NO: 3), (SEQ ID NO: 4), (SEQ ID NO: 5), (SEQ ID NO: 6), (SEQ ID NO: 7), (SEQ ID NO: 8), (SEQ ID NO: 9), (SEQ ID NO: 10), (SEQ ID NO: 11), (SEQ ID NO:
12), (SEQ ID NO: 13), (SEQ ID NO: 14), (SEQ ID NO: 15), (SEQ ID NO: 16), (SEQ ID NO:
17), (SEQ ID NO: 18), (SEQ ID NO: 19), (SEQ ID NO: 20) and (SEQ ID NO: 21).

10. The isolated polynucleotide of claim 9, wherein the nucleotide sequence is selected from the group consisting of (SEQ ID NO: 3), (SEQ ID NO: 4), (SEQ ID NO: 7), (SEQ ID NO: 8), (SEQ ID NO: 11), (SEQ ID NO: 12), (SEQ ID NO: 15), (SEQ ID NO: 16), (SEQ ID NO: 20) and (SEQ ID NO: 21).

11. The isolated polynucleotide of claim 9 wherein the polynucleotide is conjugated to a detectable label comprising a 5′-fluorophore/3′-quencher selected from Cal Red 610/BHQ-2, Quasar 670/BHQ-2, FAM/BHQ-1, Cal Orange 560/BHQ-1, or Cal Orange 560, BHQ-1.

12. The isolated polynucleotide according to claim 1 wherein the polynucleotide is labeled with a 5′-fluorophore/3′-quencher selected from Cal Red 610/BHQ-2, Quasar 670/BHQ-2, FAM/BHQ-1, Cal Orange 560/BHQ-1, or Cal Orange 560, BHQ-1.

13. A method for determining a CYP2C9 genotype of a subject, the method comprising:
a) contacting a probe with a sample comprising a nucleic acid having a sequence corresponding to the CYP2C9 genotype of the subject, wherein the probe comprises at least one isolated polynucleotide comprising a sequence consisting of at least 10, at least 18, at least 20, or at least 50 contiguous nucleotides or a complement thereof, wherein the contiguous nucleotides are contained in a nucleotide sequence selected from the group consisting of: (SEQ ID NO: 3), (SEQ ID NO: 4), (SEQ ID NO: 7), (SEQ ID NO: 8), (SEQ ID NO: 20), and (SEQ ID NO: 21); and
b) determining the CYP2C9 genotype of the subject, wherein selective hybridization of the probe to the nucleic acid is indicative of the CYP2C9 genotype.

14. The method of claim 13, wherein the CYP2C9 genotype is wild-type, CYP2C9*2, CYP2C9*3, or a combination thereof.

15. The method of claim 13, wherein the nucleic acid is an amplicon.

16. The method of claim 13, wherein the subject is a human.

17. The method of claim 13, wherein the method further comprises amplifying the nucleic acid having the sequence corresponding to the CYP2C9 genotype of the subject using at least one primer pair, wherein the at least one primer pair comprises a first primer having a first primer nucleotide sequence and a second primer having a second primer nucleotide sequence, wherein the first and the second primer nucleotide sequence is selected from the group consisting of
(SEQ ID NO: 1) and (SEQ ID NO: 2);
(SEQ ID NO: 1) and (SEQ ID NO: 17);
(SEQ ID NO: 1) and (SEQ ID NO: 19);
(SEQ ID NO: 5) and (SEQ ID NO: 6);
(SEQ ID NO: 9) and (SEQ ID NO: 10);
(SEQ ID NO: 13) and (SEQ ID NO: 14);
(SEQ ID NO: 18) and (SEQ ID NO: 17); and
(SEQ ID NO:18) and (SEQ ID NO: 19).

18. The method of claim 17, wherein the amplifying comprises performing a PCR.

19. The method of claim 18, wherein the PCR is real-time PCR.

20. A method for determining a VKORC1 genotype of a subject, the method comprising:
a) contacting a probe with a sample comprising a nucleic acid having a sequence corresponding to the VKORC1 genotype of the subject, wherein the probe comprises at least one isolated polynucleotide comprising a nucleotide sequence or complement thereof, wherein the nucleotide sequence is selected from the group consisting of (SEQ ID NO: 11), (SEQ ID NO: 12), (SEQ ID NO: 15), and (SEQ ID NO: 16), wherein the isolated polynucleotide has a total nucleotide length of about 18 to about 50 nucleotides;
b) determining the VKORC1 genotype of the subject, wherein selective hybridization of the probe to the nucleic acid is indicative of the VKORC1 genotype.

21. The method of claim 20, wherein the VKORC1 genotype is wild-type, 1173 C>T variation, 3730 G>A variation, or a combination thereof.

22. The method of claim 20, wherein the nucleic acid is an amplicon.

23. The method of claim 20, wherein the subject is a human.

24. The method of claim 20, wherein the method further comprises amplifying the nucleic acid having the sequence corresponding to the VKORC1 genotype of the subject using at least one primer pair, wherein the at least one primer pair comprises a first primer having a first primer nucleotide sequence and a second primer having a second primer nucleotide sequence, wherein the first and the second primer nucleotide sequence is selected from the group consisting of: (SEQ ID NO: 9) and (SEQ ID NO: 10); and (SEQ ID NO: 13) and (SEQ ID NO: 14).

25. The method of claim 24, wherein the amplifying comprises a PCR.

26. The method of claim 25, wherein the PCR is real-time PCR.

27. A method for selecting a medication or an optimal dose of a medication for a subject, the method comprising:
a) genotyping CYP2C9, VKORC1, or both to determine a genotype and, optionally, genotyping at least one additional gene; and
b) selecting the medication or the optimal dose of the medication based on the genotyping of step a).

28. The method of claim 27, wherein the genotype comprises wild-type CYP2C9, CYP2C9*2, CYP2C9*3, wild-type VKORC1, VKORC1 1173 C>T, VKORC1 3730 G>A, or a combination thereof.

29. A method for selecting an optimal dose of a medication for a human subject, the method comprising:
a) genotyping CYP2C9, VKORC I, or both to determine a genotype and, optionally, genotyping at least one additional gene; and
b) selecting the optimal dose of the medication based on the genotyping of step a), wherein the selecting further comprises using an algorithm based on the subject's CYP2C9 and/or VKORC1 genetic polymorphism, and one or more characteristics of the subject.

30. The method of claim 29, wherein the medication is warfarin.

31. A method for determining an optimal dose of warfarin for a human subject, the method comprising:
a) genotyping CYP2C9, VKORC1, or both to determine a genotype and, optionally, genotyping at least one additional gene; and
b) selecting the optimal dose of warfarin based on the genotyping of step a), wherein the selecting further comprises using an algorithm based on the subject's CYP2C9 and/or VKORC1 genetic polymorphism, and one or more characteristics of the subject.

32. The method of claim 31, wherein the algorithm comprises an equation, wherein the equation is √Dose (i.e., square root of dose)=0.628−0.0135(age in years)−0.240(CYP*2)−0.370(CYP*3)−0.241(VKORC1 1173)+0.24(VKORC1 3730)+0.0162(height in centimeters), wherein input values for CYP*2 and CYP*3 genotype are 0, 1 or 2 according to the number of CYP*2 or CYP*3 alleles present wherein input is 1 for VKORC1 1173CC, 2 for VKORC1 1173CT, and 3 for VKORC1 1173TT, wherein input is 0 for VKORC1 3730GG, 0 for VKORC1 3730GA, and 1 for VKORC1 3730AA.

33. An article of manufacture comprising one or more of the polynucleotides according to claim 1.

34. The article of manufacture of claim 33 further comprising reagents necessary for PCR.

35. The article of manufacture of claim 33, wherein the polynucleotide is conjugated to a detectable label comprising a 5′-fluorophore/3′-quencher selected from Cal Red 610/BHQ-2, Quasar 670/BHQ-2, FAM/BHQ-1, Cal Orange 560/BHQ-1, or Cal Orange 560, BHQ-1.

36. An article of manufacture comprising one or more of the polynucleotides according to claim 1 wherein the one or more of the polynucleotides are immobilized on a substrate.


SEQUENCE LISTING (37 sequences)

SEQ ID NO: 1 - DNA, 25 bp, Artificial Sequence
  Description of Artificial Sequence Synthetic primer
cccctgaattgctacaacaaatgtg

SEQ ID NO: 2 - DNA, 22 bp, Artificial Sequence
  Description of Artificial Sequence Synthetic primer
gacttcgaaaacatggagtgca

SEQ ID NO: 3 - DNA, 19 bp, Artificial Sequence
  Description of Artificial Sequence Synthetic probe
cagagataccttgaccttc

SEQ ID NO: 4 - DNA, 20 bp, Artificial Sequence
  Description of Artificial Sequence Synthetic probe
ccagagatacattgaccttc

SEQ ID NO: 5 - DNA, 20 bp, Artificial Sequence
  Description of Artificial Sequence Synthetic primer
ctcatgacgctgcggaattt

SEQ ID NO: 6 - DNA, 32 bp, Artificial Sequence
  Description of Artificial Sequence Synthetic primer
gaagatagtagtccagtaaggtcagtgatatg

SEQ ID NO: 7 - DNA, 18 bp, Artificial Sequence
  Description of Artificial Sequence Synthetic probe
cattgaggaccgtgttca

SEQ ID NO: 8 - DNA, 19 bp, Artificial Sequence
  Description of Artificial Sequence Synthetic probe
cattgaggactgtgttcaa

SEQ ID NO: 9 - DNA, 19 bp, Artificial Sequence
  Description of Artificial Sequence Synthetic primer
atcctgacgtggccaaagg

SEQ ID NO: 10 - DNA, 21 bp, Artificial Sequence
  Description of Artificial Sequence Synthetic primer
ccacctgggctatcctctgtt

SEQ ID NO: 11 - DNA, 24 bp, Artificial Sequence
  Description of Artificial Sequence Synthetic probe
ccaggagatcatcgacccttggac

SEQ ID NO: 12 - DNA, 28 bp, Artificial Sequence
  Description of Artificial Sequence Synthetic probe
ccaggagatcatcgactcttggactagg

SEQ ID NO: 13 - DNA, 25 bp, Artificial Sequence
  Description of Artificial Sequence Synthetic primer
ccctagatgtggggcttctagatta

SEQ ID NO: 14 - DNA, 19 bp, Artificial Sequence
  Description of Artificial Sequence Synthetic primer
agcgtgtggcacatttggt

SEQ ID NO: 15 - DNA, 26 bp, Artificial Sequence
  Description of Artificial Sequence Synthetic probe
ctcctgccatacccacacatgacaat

SEQ ID NO: 16 - DNA, 22 bp, Artificial Sequence
  Description of Artificial Sequence Synthetic probe
ctcctgccatacccgcacatga

SEQ ID NO: 17 - DNA, 23 bp, Artificial Sequence
  Description of Artificial Sequence Synthetic primer
gacttcgaaaacatggagttgca

SEQ ID NO: 18 - DNA, 20 bp, Artificial Sequence
  Description of Artificial Sequence Synthetic primer
tgcaagacaggagccacatg

SEQ ID NO: 19 - DNA, 26 bp, Artificial Sequence
  Description of Artificial Sequence Synthetic primer
ttaccttgggaatgagatagtttctg

SEQ ID NO: 20 - DNA, 23 bp, Artificial Sequence
  Description of Artificial Sequence Synthetic probe
tccagagataccttgaccttctc

SEQ ID NO: 21 - DNA, 23 bp, Artificial Sequence
  Description of Artificial Sequence Synthetic probe
tccagagatacattgaccttctc

SEQ ID NO: 22 - DNA, 180 bp, Homo sapiens
tggaaggagatccggcgtttctccctcatgacgctgcggaattttgggatggggaagaggagcattgaggaccgtgttcaagaggaagcccgctgccttgtggaggagttgagaaaaaccaagggtgggtgaccctactccatatcactgaccttactggactactatcttctctactga

SEQ ID NO: 23 - DNA, 360 bp, Homo sapiens
gataccttcatgattcatatacccctgaattgctacaacaaatgtgccatttttctccttttccatcagtttttacttgtgtcttatcagctaaagtccaggaagagattgaacgtgtgattggcagaaaccggagcccctgcatgcaagacaggagccacatgccctacacagatgctgtggtgcacgaggtccagagatacattgaccttctccccaccagcctgccccatgcagtgacctgtgacattaaattcagaaactatctcattcccaaggtaagtttgtttctcctacactgcaactccatgttttcgaagtccccaaattcatagtatcatttttaaacctctaccatca

SEQ ID NO: 24 - DNA, 117 bp, Homo sapiens
atagggtcagtgacatggaatcctgacgtggccaaaggtgcccggtgccaggagatcatcgacccttggactaggatgggaggtcggggaacagaggatagcccaggtggcttcttg

SEQ ID NO: 25 - DNA, 180 bp, Homo sapiens
tttgctttggcatgtgagccttgcctaagggggcatatctgggtccctagaaggccctagatgtggggcttctagattaccccctcctcctgccatacccgcacatgacaatggaccaaatgtgccacacgctcgctcttttttacacccagtgcctctgactctgtccccatgggctgg

SEQ ID NO: 26 - DNA, 10 bp, Artificial Sequence
  Description of Artificial Sequence Synthetic oligonucleotide
aggaccgtgt

SEQ ID NO: 27 - DNA, 13 bp, Artificial Sequence
  Description of Artificial Sequence Synthetic oligonucleotide
aggacygtgttca

SEQ ID NO: 28 - DNA, 11 bp, Artificial Sequence
  Description of Artificial Sequence Synthetic oligonucleotide
tgaggactgtg

SEQ ID NO: 29 - DNA, 13 bp, Artificial Sequence
  Description of Artificial Sequence Synthetic oligonucleotide
atacattgacctt

SEQ ID NO: 30 - DNA, 13 bp, Artificial Sequence
  Description of Artificial Sequence Synthetic oligonucleotide
agatacmttgacc

SEQ ID NO: 31 - DNA, 14 bp, Artificial Sequence
  Description of Artificial Sequence Synthetic oligonucleotide
atacattgascttc

SEQ ID NO: 32 - DNA, 13 bp, Artificial Sequence
  Description of Artificial Sequence Synthetic oligonucleotide
actcmagggtcga

SEQ ID NO: 33 - DNA, 10 bp, Artificial Sequence
  Description of Artificial Sequence Synthetic oligonucleotide
cmagrgtcga

SEQ ID NO: 34 - DNA, 11 bp, Artificial Sequence
  Description of Artificial Sequence Synthetic oligonucleotide
agagtcgatga

SEQ ID NO: 35 - DNA, 11 bp, Artificial Sequence
  Description of Artificial Sequence Synthetic oligonucleotide
ccatacccgca

SEQ ID NO: 36 - DNA, 13 bp, Artificial Sequence
  Description of Artificial Sequence Synthetic oligonucleotide
tacccrcacatga

SEQ ID NO: 37 - DNA, 12 bp, Artificial Sequence
  Description of Artificial Sequence Synthetic oligonucleotide
tacccacacatg